(19) United States
(12) Patent Application Publication
(10) Pub. No.: US 2009/0253745 A1
(43) Pub. Date: Oct. 8, 2009

(54) MODULATORS OF OCULAR OXIDATIVE STRESS

(75) Inventors: Nathan L. MATA, San Diego, CA (US); Kim B. PHAN, San Diego, CA (US); Yun HAN, San Diego, CA (US); Tam V. BUI, Laguna Niguel, CA (US); Mustapha HADDACH, San Diego, CA (US)

Correspondence Address: WILSON SONSINI GOODRICH & ROSATI, 650 PAGE MILL ROAD, PALO ALTO, CA, 94304-1050, US

(73) Assignee: SIRION THERAPEUTICS, INC., Tampa, FL (US)

(21) Appl. No.: 12/324,599

(22) Filed: Nov. 26, 2008

Related U.S. Application Data
(60) Provisional application No. 60/990,885, filed on Nov. 28, 2007.

(51) Int. Cl. A61K 31/4545; C07D 401/14; C07D 211/94; C07D 401/12; C07D 413/14; C07D 417/14; A61K 31/45; A61P 27/02

(52) U.S. Cl. 514/318; 546/193; 546/242; 514/315

(57) ABSTRACT

Described herein are compounds, compositions and methods directed to the treatment of ophthalmic conditions characterized by oxidative stress or damage in a subject by reducing the reactive oxygen species in the subject. Also described herein are methods for reducing ophthalmic photooxidative damage in a subject.

### RELATED APPLICATIONS

This application claims the benefit of U.S. Provisional Patent Application No. 60/990,885, filed Nov. 28, 2007, the contents of which are incorporated by reference in its entirety.

### FIELD OF THE INVENTION

The methods, compounds and compositions described herein are directed to the treatment of ophthalmic conditions characterized by oxidative stress or damage in a subject by reducing reactive oxygen species in the subject.

### BACKGROUND OF THE INVENTION

Oxidative damage plays a causative or contributing role in the pathogenesis of many diseases, such as heart disease, certain types of cancers, neurodegenerative disorders, ocular and age-related diseases. Although oxygen is necessary for life in providing aerobic respiration, an accumulation of free radicals or reactive oxygen species (ROS) can cause oxidative damage to cells and tissues. ROS such as superoxide, hydrogen peroxide and singlet oxygen may and can cause lipid peroxidation, protein oxidation and mutagenesis, which damage the eye, such as the retinal pigment epithelium or Bruch's membrane. Accumulation of ROS-induced oxidative damage contributes to age-related eye diseases such as macular degeneration, glaucoma, cataracts, and other eye diseases. Diabetes, smoking, exposure to excessive sunlight, and ozone also contribute to oxidative stress.

The degree of oxidative damage is restricted by free radical scavengers and antioxidants and the repair of damaged elements. Free radical scavengers and antioxidants may act at different levels in the oxidation process, for example, by preventing formation of initiating radicals, binding metal ions or removing damaged molecules.

### SUMMARY OF THE INVENTION

Presented herein are methods, compounds, and compositions for the treatment of ophthalmic conditions characterized by oxidative stress or damage comprising administering to a subject in need a compound having an N-oxyl moiety (used interchangeably herein with nitroxide moiety or aminooxyl moiety). In one aspect, the ophthalmic condition characterized by oxidative stress or damage is a vitreoretinal disease or condition. In other aspects, the ophthalmic condition is diabetic retinopathy or age-related macular degenerations. Also presented herein are methods for reducing or preventing ophthalmic photooxidative damage in a subject comprising administering to a subject in need a compound having a N-oxyl moiety. In one embodiment of any of the aforementioned methods and compositions, the compound having a N-oxyl moiety is ophthalmically administered to the subject in need.

In one aspect are compounds of Formula I or a pharmaceutically acceptable solvate or a pharmaceutically acceptable salt thereof:

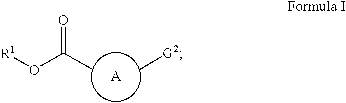

wherein,
R¹ is N-oxyl-2,2,6,6-tetramethylpiperidin-4-yl or N-oxyl-2,2,5,5-tetramethylpyrrolidin-3-yl;

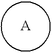

is an optionally substituted 5-membered heterocycle containing at least 1 N atom in the heterocyclic ring, or an optionally substituted 6-membered heterocycle containing at least 1 N atom in the heterocyclic ring; and
G² is selected from H, C₁-C₆ alkyl, —CF₃, —CN, —CO₂H, —CO₂R², tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄alkyl group or an optionally substituted phenyl group;
each R³ is independently selected from H, an optionally substituted C₁-C₄ alkyl group, an optionally substituted aryl, and an optionally substituted heteroaryl.

In a further embodiment, R¹ is N-oxyl-2,2,6,6-tetramethylpiperidin-4-yl. In a further or alternative embodiment,

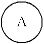

is an optionally substituted 5-membered heteroaryl containing at least 1 N atom in the heteroaryl ring or an optionally substituted 6-membered heteroaryl containing at least 1 N atom in the heteroaryl ring. In yet a further or alternative embodiment, G² is H, methyl, ethyl, CF₃, CN, CO₂H, CO₂Me, CO₂Et, tetrazolyl, —NHS(═O)₂Me, —NHS(═O)₂Ph, —S(═O)₂NH₂, S(═O)₂NHMe, OH, —OMe, —C(═O)CF₃, —C(O)NHS(═O)₂Me, —S(═O)₂NHC(═O)Me, optionally substituted aryl, or an optionally substituted heteroaryl. In still a further or alternative embodiment,

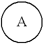

is an optionally substituted group selected from pyrazolylene, isoxazolylene, isothiazolylene, pyrrolylene, oxazolylene, thiazolylene, imidazolylene, pyridinylene, pyrimidinylene and pyrazinylene. In still a further or alternative embodiment, G² is selected from an optionally substituted aryl and an optionally substituted heteroaryl. In still a further or alternative embodiment, G² is selected from an optionally substituted phenyl and an optionally substituted heteroaryl containing at least 1 N atom in the heteroaryl ring. In still a further or alternative embodiment,

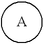

is an optionally substituted group selected from pyrazolylene, isoxazolylene, isothiazolylene, pyrrolylene, oxazolylene, thiazolylene, and imidazolylene. In still a further or alternative embodiment, G² is an optionally substituted group selected from phenyl, pyrazolyl, imidazolyl, pyridinyl, pyrimidinyl and pyrazinyl. In still a further or alternative embodiment,

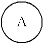

is an optionally substituted group selected from pyrazolylene, isoxazolylene, and isothiazolylene. In still a further or alternative embodiment, G² is an optionally substituted phenyl or pyridinyl.

In a further or alternative embodiment, the compound of Formula I has the structure of Formula II:

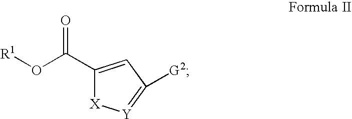

wherein;

X is O, S, NH or CH₂;

Y is CH or N;

R¹ is N-oxyl-2,2,6,6-tetramethylpiperidin-4-yl;

G² is a (substituted or unsubstituted aryl) or a (substituted or unsubstituted heteroaryl).

In still a further or alternative embodiment, Y is N. In still a further or alternative embodiment, X is O, S, or NH. In still a further or alternative embodiment, X is NH. In still a further or alternative embodiment, G² is a (substituted or unsubstituted phenyl) or a (substituted or unsubstituted heteroaryl containing at least 1 N atom in the heteroaryl ring). In still a further or alternative embodiment, G² is a (substituted or unsubstituted phenyl), (substituted or unsubstituted 5-membered heteroaryl containing at least 1 N atom in the heteroaryl ring), or a (6-membered heteroaryl containing at least 1 N atom in the heteroaryl ring). In still a further or alternative embodiment, G² is a substituted or unsubstituted group selected from phenyl, pyrazolyl, imidazolyl, pyridinyl, pyrimidinyl and pyrazinyl. In still a further or alternative embodiment, G² is a (substituted or unsubstituted phenyl) or a (6-membered heteroaryl containing at least 1 N atom in the heteroaryl ring). In still a further or alternative embodiment, G² is a substituted or unsubstituted group selected from phenyl, pyridinyl, pyrimidinyl and pyrazinyl.

In further or alternative embodiment, the compound is selected from:
N-oxyl-2,2,6,6-tetramethylpiperidin-4-yl 3-(phenyl)-1H-pyrazole-5-carboxylate;
N-oxyl-2,2,6,6-tetramethylpiperidin-4-yl 3-(pyridin-4-yl)-1H-pyrazole-5-carboxylate;
N-oxyl-2,2,6,6-tetramethylpiperidin-4-yl isonicotinate;
N-oxyl-2,2,6,6-tetramethylpiperidin-4-yl 5-methylpyrazine-2-carboxylate;
N-oxyl-2,2,6,6-tetramethylpiperidin-4-yl picolinate; and
N-oxyl-2,2,6,6-tetramethylpiperidin-4-yl nicotinate.

In one embodiment, the compound of Formula II has the formula:

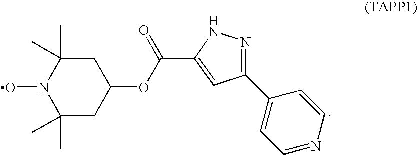

In one embodiment, described here are compounds of Formula III or pharmaceutically acceptable solvate, pharmaceutically acceptable salt, or pharmaceutically acceptable prodrug thereof:

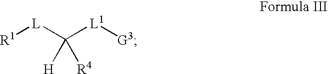

wherein,
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —OC(═O)—, —C(═O)O—, —OCH₂—, —CH₂O—, —NR⁶C(═O)—, —C(═O)NR⁶—, —NR⁶CH₂—, —CH₂NR⁶—, or an optionally substituted C₁-C₈alkylene;
L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NH S(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl;
R⁶ is H, an optionally substituted C₁-C₄ alkyl group, —C(═O)R², and —SO₂N(R³)₂.

In one embodiment are compounds of Formula IIa or a pharmaceutically acceptable solvate, or a pharmaceutically acceptable salt thereof:

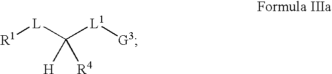

wherein,
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —OC(═O)—, —C(═O)O—, —OCH₂— or —CH₂O—;
L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl.

In a further or alternative embodiment, R¹ is N-oxyl-2,2,6,6-tetramethylpiperidin-4-yl. In still a further or alternative embodiment, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted aryl, and an optionally substituted heteroaryl. In still a further or alternative embodiment, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted phenyl, optionally substituted pyrazolyl, optionally substituted imidazolyl, optionally substituted pyridinyl, optionally substituted pyrimidinyl, and optionally substituted pyrazinyl. In still a further or alternative embodiment, G³ is selected from —CO₂H, —CO₂R², tetrazolyl, optionally substituted aryl, and an optionally substituted heteroaryl. In still a further or alternative embodiment, R⁴ is H. In still a further or alternative embodiment, L¹ is an optionally substituted C₁-C₈alkylene optionally containing at least one unit of unsaturation. In still a further or alternative embodiment, L¹ is a bond, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH═CH—, or —CH₂CH₂CH₂CH₂CH(OH)CH═CH—. In still a further or alternative embodiment, L is —OC(═O)— or —OCH₂—. In still a further or alternative embodiment, G3 is selected from —CO₂H, —CO₂R² and tetrazolyl. In still a further or alternative embodiment, R⁴ is N(R⁵)₂; and R⁵ is H. In still a further or alternative embodiment, -L¹-G³ is selected from H, —CH₃, —CH₂CH(CH₃)₂, —CH₂CO₂H, —CH₂CH₂CO₂H, —CH₂CH₂CH₂CH═CHCO₂H, —CH₂CH₂CH₂CH₂CH(OH)CH═CHCO₂H, —CH₂CONH₂, —CH₂CH₂CONH₂, —CH₂CH₂CH₂CH₂NH₂, —CH₂CH₂CH₂NC(═NH)NH₂, —CH₂OH, —CH₂CH₂SCH₃, —CH(OH)CH₃, —CH₂SH, —CH(CH₃)₂, —CH(CH₃)CH₂CH₃, —CH₂-imidazolyl, —CH₂-(1H-indol-3-yl), —CH₂-phenyl, —CH₂-(4-hydroxyphenyl). In still a further or alternative embodiment, L is —OC(═O)—.

In a further or alternative embodiment is a compound selected from:
1-N-oxyl-2,2,6,6-tetramethylpiperidin-4-yl 2-(pyridin-2-yl)acetate;
1-N-oxyl-2,2,6,6-tetramethylpiperidin-4-yl 2-amino-3-phenylpropanoate;
4-(1-N-oxyl-2,2,6,6-tetramethylpiperidin-4-yloxy)-4-yl succinate; and
(E)-9-((N-oxyl-2,2,6,6-tetramethylpiperidin-4-yloxy)carbonyl)-4-hydroxynon-2-enoic acid.

In one embodiment, the compound of Formula IIa has the structure:

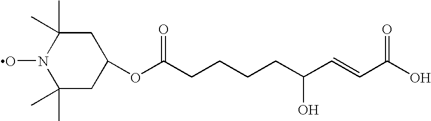

In one embodiment are compounds of Formula IIb or pharmaceutically acceptable solvate, pharmaceutically acceptable salt, or pharmaceutically acceptable prodrug thereof

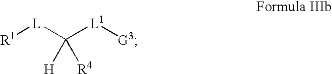

wherein,
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —NR⁶C(═O)—, —C(═O)NR⁶—, —NR⁶CH₂—, —CH₂NR⁶—, or an optionally substituted C₁-C₈alkylene;
L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl;
each R⁶ is independently selected from H, an optionally substituted C₁-C₄ alkyl group, —C(═O)R², and —S(═O)₂N(R³)₂.

In some embodiments, the compound of Formula IIb is a compound wherein R¹ is N-oxyl-2,2,6,6-tetramethylpiperidin-4-yl. In some embodiments, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted aryl, and an optionally substituted heteroaryl. In other embodiments, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted phenyl, optionally substituted pyrazolyl, optionally substituted imidazolyl, optionally substituted pyridinyl, optionally substituted pyrimidinyl, and optionally substituted pyrazinyl. In still further embodiments, G³ is selected from —CO₂H, —CO₂R², tetrazolyl, optionally substituted aryl, and an optionally substituted heteroaryl.

In some embodiments, the compound of Formula IIIb is a compound wherein R⁴ is H. In some embodiments of Formula (IIIb), L¹ is an optionally substituted C₁-C₈alkylene optionally containing at least one unit of unsaturation. In some embodiments, L¹ is a bond, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH═CH—, or —CH₂CH₂CH₂CH₂CH(OH)CH═CH—.

In some embodiments of Formula IIIb, L is —NR⁶C(═O)— or —NR⁶CH₂— or an optionally substituted C₁-C₈alkylene.

In some embodiments of Formula IIIb, G³ is selected from —CO₂H, —CO₂R² and tetrazolyl. In some embodiments, R⁴ is N(R⁵)₂; and R⁵ is H.

In some embodiments of Formula IIIb, -L¹-G³ is selected from H, —CH₃, —CH₂CH(CH₃)₂, —CH₂CO₂H, —CH₂CH₂CO₂H, —CH₂CH₂CH₂CH═CHCO₂H, —CH₂CH₂CH₂CH₂CH(OH)CH═CHCO₂H, —CH₂CONH₂, —CH₂CH₂CONH₂, —CH₂CH₂CH₂CH₂NH₂, —CH₂CH₂CH₂NC(═NH)NH₂, —CH₂OH, —CH₂CH₂SCH₃, —CH(OH)CH₃, —CH₂SH, —CH(CH₃)₂, —CH(CH₃)CH₂CH₃, —CH₂-imidazolyl, —CH₂-(1H-indol-3-yl), —CH₂-phenyl, —CH₂-(4-hydroxyphenyl).

In some embodiments of Formula IIIb, L is —NR⁶C(═O)—.

In some embodiments, the compound of Formula IIb is (E)-9-((2,2,6,6-tetramethylpiperidin-1-oxyl)-4-aminyl)-9-oxo-4-hydroxynon-2-enoic acid; (E)-9-((2,2,6,6-tetramethylpiperidin-1-hydroxide)-4-aminyl)-9-oxo-4-hydroxynon-2-enoic acid, (E)-9-((2,2,6,6-tetramethylpiperidin-1-oxyl)-4-amino-(N-acetyl))-4-hydroxynon-2-enoic acid.

In one aspect is a pharmaceutical composition comprising at least one compound of Formula I, II, IIIa, IIIb, IV or V, as described herein, and an ophthalmically acceptable excipient. In one embodiment, the composition is in the form of eye drops. In one embodiment, the composition does not further comprise a solubilizing agent. In another embodiment, the composition further comprises a solubilizing agent. In another embodiment, the solubilizing agent is selected from a cyclodextrin, a glycan, or a dextran. In another embodiment, the solubilizing agent is a sulfate of a cyclodextrin, a glycan, or a dextran. In yet another embodiment, the solubilizing agent is selected from a dextran sulfate, cyclodextrin sulfate, or β-1,3-glucan sulfate, or a derivative thereof.

In one aspect is a method for reducing ophthalmic reactive oxygen species in a subject, comprising administering to a subject a composition comprising a therapeutically effective amount of a compound of Formula I, II, IIIa, IIIb, IV or V, as described herein. In one embodiment, the subject is suffering from or at risk of suffering from an ophthalmic condition characterized by oxidative damage. In another embodiment, the ophthalmic condition is a vitreoretinal disease or condition. In yet another embodiment, the ophthalmic condition is diabetic retinopathy, wet age-related macular degeneration, dry age-related macular degeneration, Stargardt's disease, macular edema, glaucoma, ocular hypertension, cataracts, or optic neuropathy. In yet another embodiment, the subject is suffering from diabetes, hypertension, arteriosclerosis, exhibits macular drusen, or smokes tobacco. In yet another embodiment, the administration is topical on an eye, intraocular, intraorbital, ophthalmic, retrobulbar, parenteral, oral, topical, intramuscular, transdermal, sublingual, intranasal, or respiratory. In yet another embodiment, the composition is administered topically to an eye. In yet another embodiment, the compound is administered as an eye drop, eye wash, or eye ointment formulation. In yet another embodiment, the therapeutically effective amount is between 0.1 mM and 100 mM. In yet another embodiment, the method further comprises administering to the subject a therapeutically effective amount of an antioxidant, such as vitamin C, vitamin E, beta-carotene and other carotenoids, coenzyme Q, lutein, butylated hydroxytoluene, resveratrol, a trolox analogue (PNU-83836-E), bilberry extract, and zeaxanthin.

In one aspect is a method for treating an oxidative ophthalmic condition in a subject in need thereof, comprising administering to the subject a composition comprising a therapeutically effective amount of the compound of Formula I, II, IIIa, IIIb, IV or V, as described herein, wherein the ophthalmic condition is characterized by ophthalmic oxidative damage. In one embodiment, the ophthalmic condition is diabetic retinopathy, wet age-related macular degeneration, dry age-related macular degeneration, Stargardt's disease, macular edema, glaucoma, ocular hypertension, cataracts, or optic neuropathy. In another embodiment, the ophthalmic condition is diabetic retinopathy. In yet another embodiment, the ophthalmic condition is wet-age related macular degeneration or dry age-related macular degeneration. In yet another embodiment, the administration is topical on an eye, intraocular, intraorbital, ophthalmic, retrobulbar, parenteral, oral, topical, intramuscular, transdermal, sublingual, intranasal, or respiratory. In yet another embodiment, the composition is administered topically to an eye. In yet another embodiment, the compound is administered as an eye drop, eye wash, or eye ointment formulation. In yet another embodiment, the therapeutically effective amount is between 0.1 mM and 100 mM. In yet another embodiment, the method further comprises administering to the subject a therapeutically effective amount of an antioxidant, such as vitamin C, vitamin E, beta-carotene and other carotenoids, coenzyme Q, lutein, butylated hydroxytoluene, resveratrol, a trolox analogue (PNU-83836-E), bilberry extract, and zeaxanthin.

In one aspect is a method for preventing or reducing ophthalmic photooxidative damage in a subject, comprising administering to the subject a composition comprising a therapeutically effective amount of a compound of Formula I, II, IIIa, IIIb, IV or V, as described herein. In one embodiment, the composition is administered topically to an eye. In another embodiment, the subject is at high risk for an ophthalmic condition. In yet another embodiment, the subject is suffering from diabetes, hypertension, arteriosclerosis, exhibits macular drusen, or smokes tobacco. In yet another embodiment, the administration precedes exposure to sunlight and/or ultraviolet light. In yet another embodiment, the administration is topical on an eye, intraocular, intraorbital, ophthalmic, retrobulbar, parenteral, oral, topical, intramuscular, transdermal, sublingual, intranasal, or respiratory. In yet another embodiment, the composition is administered topically to an eye. In yet another embodiment, the compound is administered as an eye drop, eye wash, or eye ointment formulation. In yet another embodiment, the therapeutically effective amount is between 0.1 mM and 100 mM. In yet another embodiment, the method further comprises administering to the subject a therapeutically effective amount of an antioxidant, such as vitamin C, vitamin E, beta-carotene and other carotenoids, coenzyme Q, lutein, butylated hydroxytoluene, resveratrol, a trolox analogue (PNU-83836-E), bilberry extract, and zeaxanthin.

In another aspect is a kit comprising an eye drop formulation comprising an effective amount of a compound of Formula I, II, IIIa, IIIb, IV or V, as described herein, a dispenser, and instructions describing when and how much of the formulation should be applied to the eye.

### Certain Definitions

An “alkyl” group refers to an aliphatic hydrocarbon group. The alkyl moiety includes a “saturated alkyl” group, which means that it does not contain any units of unsaturation (e.g. carbon-carbon double bond(s) or carbon-carbon triple bond(s)). The alkyl moiety also includes an “unsaturated alkyl” moiety, which means that it contains at least one unit of unsaturation (e.g. carbon-carbon double bond(s) or carbon-carbon triple bond(s)). The alkyl moiety, whether saturated or unsaturated, includes branched, straight chain, or cyclic moieties. The point of attachment of an alkyl group is at a sp3 carbon that is not part of a ring.

The “alkyl” moiety has 1 to 10 carbon atoms (whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C1-C4 alkyl” or similar designations. By way of example only, “C1-C4 alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, 2-methyl-butyl, 2-ethyl-butyl, 3-propyl-butyl, pentyl, neo-pentyl, 2-propyl-pentyl, hexyl, propenyl, butenyl, cyclopropylmethyl, cyclobutylmethyl, cyclopentylmethyl, cyclohexylmethyl and the like. Alkyl groups include substituted or unsubstituted moieties.

As used herein, C₁-Cₓ includes C₁-C₂, C₁-C₃ . . . C₁-Cₓ. C₁-Cₓ refers to the number of carbon atoms that make up the moiety to which it designates (excluding optional substitutents).

An “alkoxy” group refers to a (alkyl)O— group, where alkyl is as defined herein.

The term “alkylamine” refers to the —N(alkyl)ₙH_{y} group, where x and y are selected from the group x=1, y=1 and x=2, y=0. When x=2, the alkyl groups, taken together, optionally form a cyclic ring system.

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). An amide includes an amino acid or a peptide molecule attached to a compound of Formula I, II, IIIa, IIIb, IV or V, thereby forming a prodrug.

The term “aromatic” refers to a planar ring having a delocalized n-electron system containing 4n+2π electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups are optionally substituted. Examples of aryl groups include, but are not limited to phenyl, and naphthalenyl. Depending on the structure, an aryl group can be a monoradical or a diradical (i.e., an arylene group).

The term “bond” or “single bond” refers to a chemical bond between two atoms, or two moieties when the atoms joined by the bond are considered to be part of larger substructure.

“Carboxy” refers to —CO₂H or a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

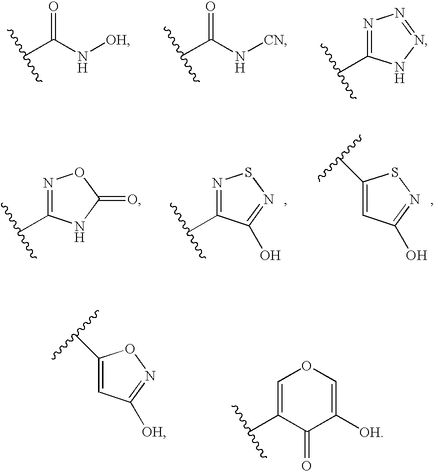

and the like.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles are optionally substituted.

The term “cycloalkyl” refers to a monocyclic or polycyclic aliphatic, non-aromatic radical, wherein each of the atoms forming the ring (i.e. skeletal atoms) is a carbon atom. Cycloalkyls include saturated, or partially unsaturated moieties. Cycloalkyls include moieties fused with an aromatic ring, and the point of attachment is at a carbon that is not an aromatic ring carbon atom. Cycloalkyl groups include groups having from 3 to 10 ring atoms. Illustrative examples of cycloalkyl groups include, but are not limited to, the following moieties:

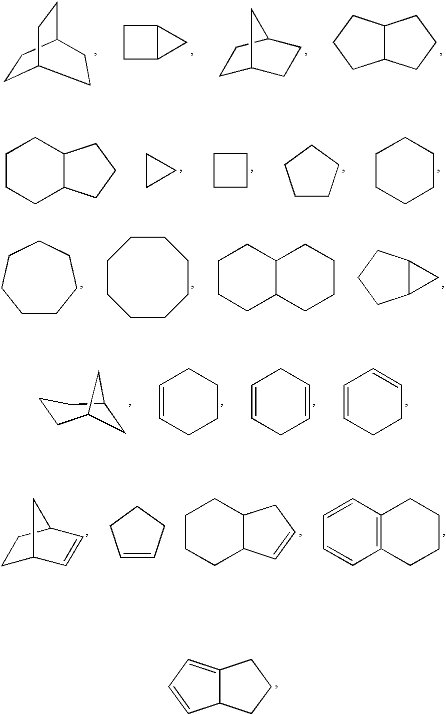

and the like. In some embodiments, cycloalkyl groups are selected from among cyclopropyl, cyclobutyl, cyclopentyl, cyclohexyl, cycloheptyl, and cyclooctyl. Cycloalkyl groups include substituted or unsubstituted moieties.

The term “cycloalkenyl” refers to a type of cycloalkyl group that contains at least one carbon-carbon double bond in the ring and where the cycloalkenyl is attached at one of the carbon atoms of the carbon-carbon double bond. Non-limiting examples of a cycloalkenyl alkenyl group include cyclopenten-1-yl, cyclohexen-1-yl, cyclohepten-1-yl, and the like. Cycloalkenyl groups include substituted or unsubstituted moieties.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified.

“Halo”, “halide”, or “halogen” refer to fluorine, chlorine, bromine, and iodine.

The term “heteroalkyl” refers to alkyl radicals that have one or more skeletal chain atoms selected from an atom other than carbon, e.g., oxygen, nitrogen, sulfur, phosphorus or combinations thereof. The heteroatom(s) are optionally placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, in some embodiments, up to two heteroatoms are consecutive, such as, by way of example, —CH₂—NH—OCH₃ and —CH₂—O—Si(CH₃)₃. Excluding the number of heteroatoms, a “heteroalkyl” has from 1 to 6 carbon atoms.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Also includes are N-containing heteroaryl that are oxidized to the corresponding N-oxide. The polycyclic heteroaryl group includes fused or non-fused moieities. Illustrative examples of heteroaryl groups include the following moieties:

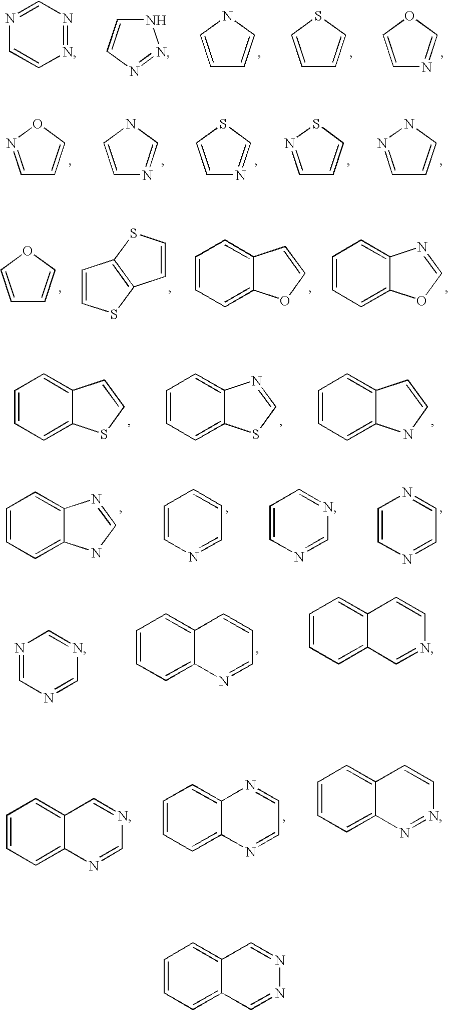

and the like.

The term “heterocycle” refers to heteroaromatic and heteroalicyclic groups (heterocycloalkyl groups) containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, include C-attached or N-attached where such is possible. For instance, a group derived from pyrrole includes pyrrol-1-yl (N-attached) or pyrrol-3-yl (C-attached). Further, a group derived from imidazole includes midazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

A “heteroalicyclic” or “heterocycloalkyl” group refers to a cycloalkyl group that includes at least ring atom selected from nitrogen, oxygen and sulfur (i.e. at least one ring atom is a heteroatom). The radicals are optionally fused with an aryl or heteroaryl. Illustrative examples of heterocycloalkyl groups, also referred to as non-aromatic heterocycles, include:

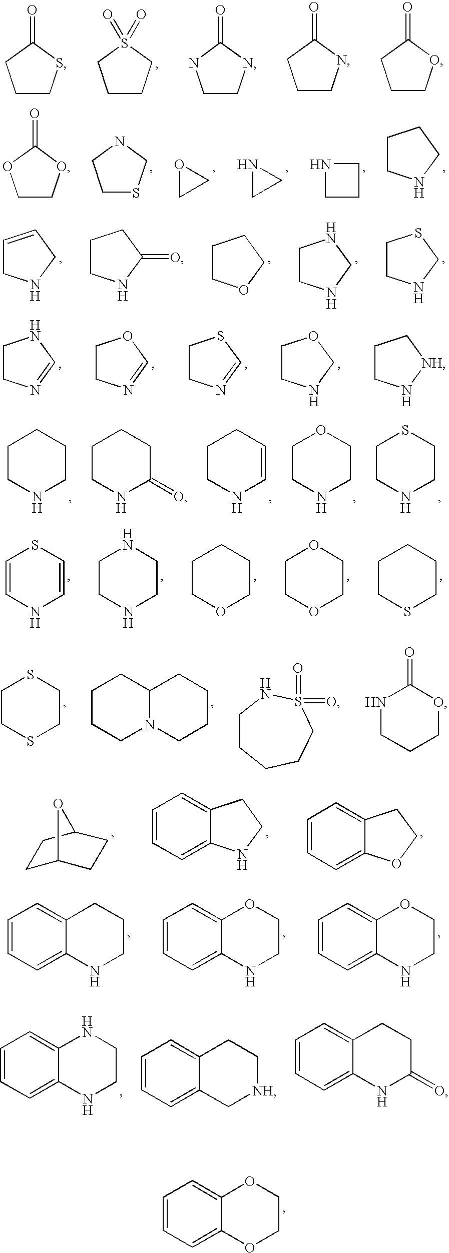

and the like. The term heterocycloalkyl also includes all ring forms of the carbohydrates, including but not limited to the monosaccharides, the disaccharides and the oligosaccharides. Other examples of heterocycloalkyls include, quinolizine, dioxine, piperidine, morpholine, thiazine, tetrahydropyridine, piperazine, oxazinanone, dihydropyrrole, dihydroimidazole, tetrahydrofuran, tetrahydropyran, dihydrooxazole, oxirane, pyrrolidine, pyrazolidine, imidazolidinone, pyrrolidinone, dihydrofuranone, dioxolanone, thiazolidine, piperidinone, tetrahydroquinoline, tetrahydrothiophene, and thiazepane. The point of attachment of a heterocycloalkyl group is at a heteroatom or carbon atom that is not part of an aromatic ring.

The term “membered ring” can embrace any cyclic structure. The term “membered” is meant to denote the number of skeletal atoms that constitute the ring. Thus, for example, cyclohexyl, pyridinyl, pyranyl and thiopyranyl are 6-membered rings and cyclopentyl, pyrrolyl, furanyl, and thienyl are 5-membered rings.

The term “N-oxyl-2,2,6,6-tetramethylpiperidin-4-oximyl” refers to a moiety having the structure:

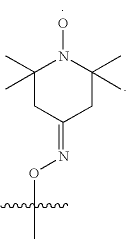

The term “N-oxyl-2,2,6,6-tetramethylpiperidin-4-yl” refers to a moiety having the structure:

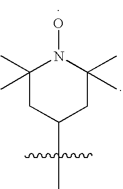

The term “N-oxyl-2,2,5,5-tetramethylpyrrolidin-3-yl” refers to a moiety having the structure:

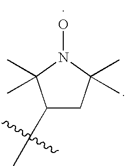

The term “optionally substituted” or “substituted” means that the referenced group include those options substituted with one or more additional group(s) individually and independently selected from alkyl, cycloalkyl, heterocycloalkyl, aryl, heteroaryl, benzyl, heteroarylmethyl, hydroxy, alkoxy, fluoroalkoxy, aryloxy, thiol, alkylthio, arylthio, alkylsulfoxide, arylsulfoxide, alkylsulfone, arylsulfone, cyano, halo, carboxy, nitro, haloalkyl, fluoroalkyl, and amino, including mono- and di-alkyl amino groups, and the protected derivatives thereof. By way of example an optional substituent is LₛRₛ, wherein LₛRₛ is halo, amino, nitro, cyano, or each L, is independently selected from a bond, —O—, —C(═O)—, —C(═O)O—, —OC(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, and C₁-C₆alkyl; and each Rₛ is independently selected from H, alkyl, fluoroalkyl, cycloalkyl, heteroaryl, aryl, benzyl, heteroarylmethyl, or heteroalkyl. By way of example an optional substituents is alkyl, hydroxy, alkoxy, fluoroalkoxy, cyano, halo, carboxy, haloalkyl, fluoroalkyl.

### BRIEF DESCRIPTION OF THE FIGURES

FIG. 1. In vitro anti-oxidative potency of compositions from compounds of Formula I, II and IIIa and IIIb. This assay relies on the ability of the test compound to inhibit the oxidation of ABTS (2,2′,azino-di-[3-ethylbenzthiazoline-6-sulfonate]) to ABTS^{•+} radical cation by metmyoglobin. Experiments were performed according to procedure provided by manufacture. Briefly, 50 μM of compound of Formula I, II, IIIa or IIIb were mixed at room temperature with a solution containing ABTS and metmyoglobin. Hydrogen peroxide was then added to activate metmyglobin to ferrylmyoglobin radical, which in turn oxidizes ABTS to form ABTS^{•+}. The oxidized form of ABTS produces a green color which absorbs at 405 nm and 750 nm. Absorption at 750 nm was monitored over time for samples in the presence or absence of compositions from compounds of Formula I, II, IIIa or IIIb. Relative anti-oxidant activity was determined by comparing absorbance values in the absence of the test compound (control), which is taken as 0% anti-oxidant potency, to the absorbance in the presence of the test compound. Representative oxidized and reduced forms of compositions from compounds of Formula I, II, IIIa and IIIb were analyzed. “O” represents the oxidized form which contains a radical at the N-oxyl position. “R” represents the reduced N-hydroxyl form. Compounds 1-5 are oxidized and reduced compositions from compounds of Formula II. Compound 6 is an oxidized and reduced composition from compounds of Formula I. Compound 7 is an oxidized and reduced composition from compounds of Formula IIIa. The data show that each of the test compounds possesses significant anti-oxidant activity.

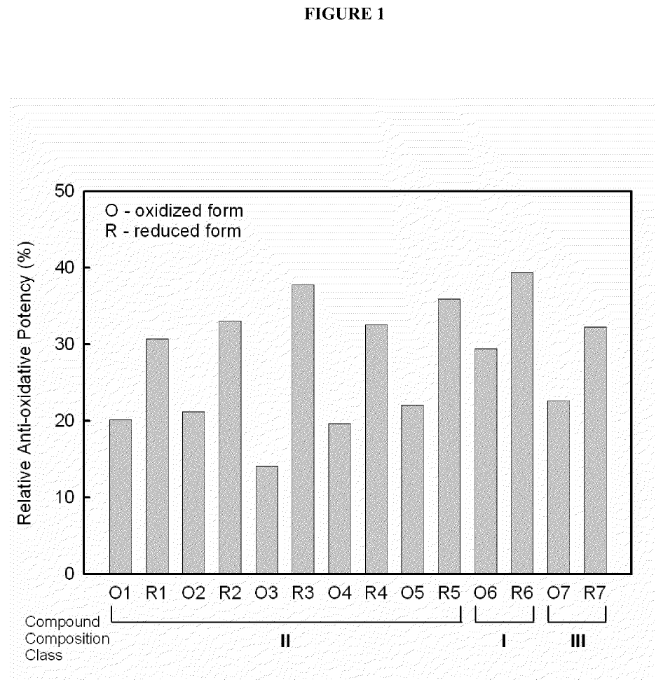

FIG. 2. In vitro hydrolysis of TAPP1 by anterior segments from mouse eyeglobes. Anterior segments were isolated from twelve wild-type mouse eyeglobes, and cultured in 0.5 ml of MEM media. TAPP1 was added to the culture to a final concentration of 1 mM, and the sample was incubated at 37° C. At 0, 1, 5, 15, 30, 60 and 120 min following incubation, 20 μl aliquot samples were removed from the culture. The aliquots were mixed with equal volume of ice-cold methanol, and incubated on ice for 10 min, followed by centrifugation at 25,000 g to precipitate proteins. TAPP1 content in the supernatant was analyzed by a capillary reverse phase C18 column. The relative quantity of TAPP1 was determined by integration of chromatographic peak area. The results show that anterior segments prepared from mouse eyeglobes rapidly hydrolyze TAPP1 (half-life ˜10 min under the experimental conditions). Release of the two products of TAPP1 hydrolysis (designated HP-1 and HP-2) was inversely proportional to the rate of TAPP1 hydrolysis (not shown). Corneal esterases may be largely responsible for the hydrolysis of TAPP1.

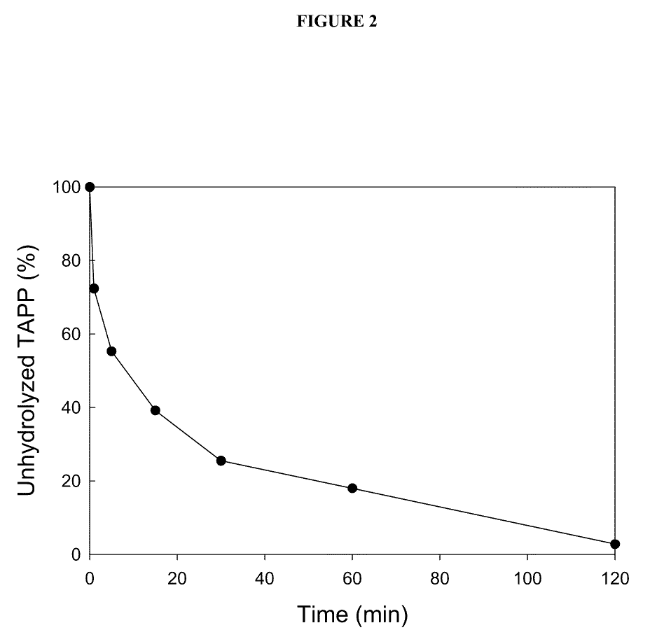

FIG. 3. Ex vivo lens toxicity test of TAPP1. The eyeglobes of mice (aged 42 days) were cultured overnight either with control solution (media containing 45% β-cyclodextrin) or TAPP1 (TAPP1 in media containing 45% β-cyclodextrin), at 37° C. overnight, and in 95% air/5% CO2. Brief treatment (10 min) of the eyeglobe with 100% ethanol caused cataract formation. However, no cataract or other overt toxicity was observed following overnight incubation of eyeglobes with TAPP1 (FIG. 3a). To determine the effects of TAPP1 on isolated lenses, intact lenses were dissected from wild-type mouse eyeglobes, and incubated in DMEM media in a 96 well tissue culture plate. Groups of 3-6 lenses were incubated in 200 μl of media alone (negative control), 4% (2-hydroxypropyl)-β-cyclodextrin (carrier control), 8 mM hydrogen peroxide (H₂O₂, positive control for lens toxicity), 4 mM TAPP1-O (free radical form), 4 mM TAPP1-R (reduced form), and 4 mM of the two products of TAPP1-R hydrolysis (HP-1 and HP-2), respectively. β-cyclodextrin was included as a carrier control because all of the stock solutions contained 45% (2-hydroxypropyl)-β-cyclodextrin. The samples were incubated in 5% CO₂ incubator at 37 C for 2 days. A representative lens from each group following the 2 day incubation is shown in the figure. While 8 mM H₂O₂ caused obvious cataract, neither TAPP1-O, TAPP1-R, HP-1 or HP-2 caused overt toxicity. Compounds from the TAPP compositions demonstrated a protective effect in maintaining the lens transparency compared to media and β-cyclodextrin controls (FIG. 3b).

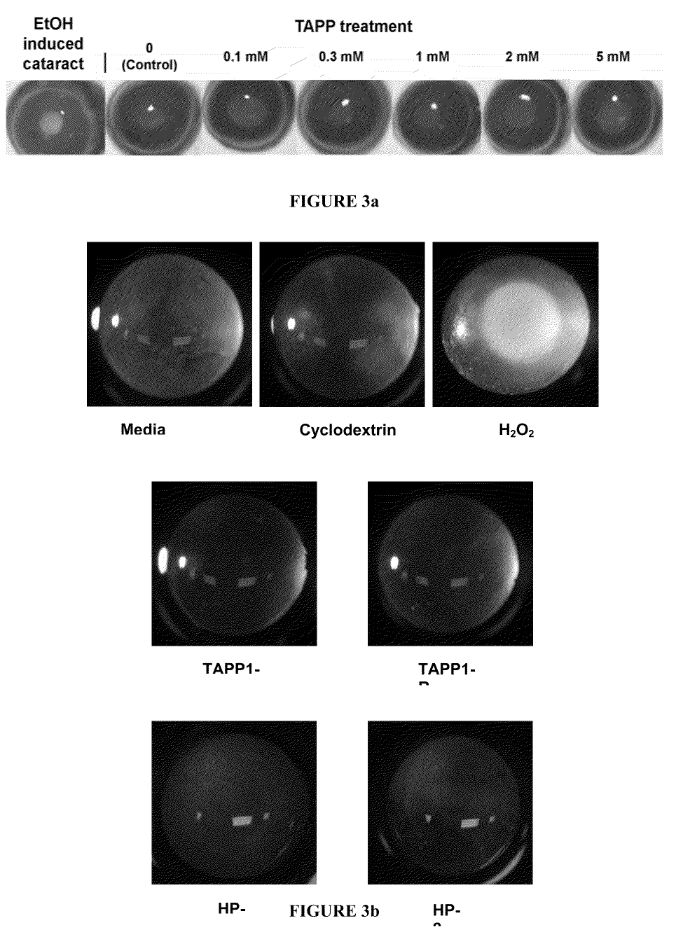

FIG. 4. In vitro anti-oxidative potency of products from hydrolyzed TAPP1. The rapid hydrolysis of TAPP1 by anterior segments prompted an analysis of the anti-oxidative potency of products from hydrolyzed TAPP1. The assay employed to measure anti-oxidative potency of these products (designated HP-1 and HP-2) has been previously described (see legend of FIG. 1). In this study, the relative anti-oxidative potency of HP-1 and HP-2 were compared to that of the reduced and oxidized forms of TAPP1 (TAPP1-R and TAPP1-O, respectively). The anti-oxidative effect demonstrated by TAPP1-R is taken as 100% anti-oxidative potency. The data show a pronounced anti-oxidative potency of HP-1; the anti-oxidative potency of HP-1 is comparable to that of TAPP1-R. Compositional analysis of HP-1 by tandem MS/MS revealed the presence of a 2,2,6,6-tetramethypiperidinoxy chemical moiety (not shown).

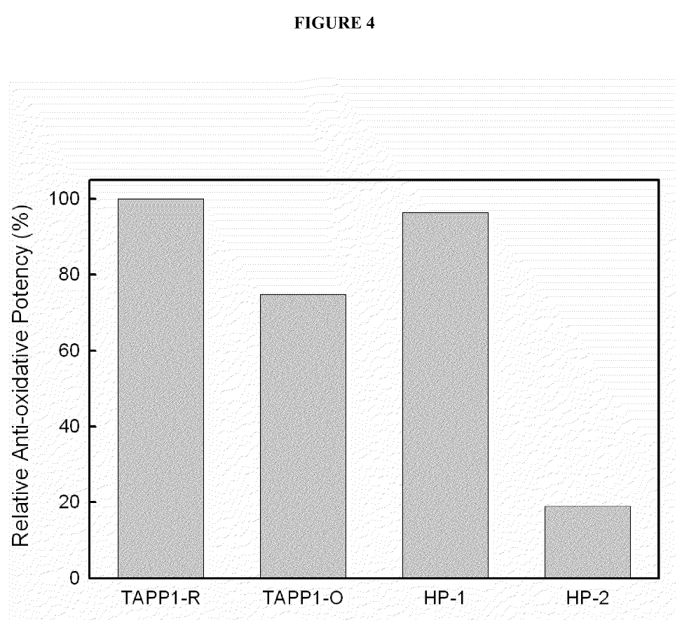

FIG. 5. In vitro analysis of potential liver toxicity of the products from TAPP1 hydrolysis. In this study, toxicity was assessed by the ability of HP-1 and HP-2 to interfere with the activities of three abundant cytochrome P450 (CYP450) isozymes (CYP2D6, CYP2E1 and CYP3A4) which are known to metabolize a wide range of bioactive pharmaceuticals. Experiments were performed using procedures provided by the manufacture (Sigma Chemical Company, St. Louis, Mo.). HP1 or HP2 (2 μM each) were pre-incubated with the CYP450 enzymes at room temperature for 20 min or longer. An enzyme specific substrate which contained a fluorescence dye coupled with a quencher was added to the mixture. CYP450 enzymes metabolized the substrate which liberated the fluorescent dye. Fluorescence intensity was monitored over time. Inhibitory effect of HP-1 or HP-2 was calculated from the relative fluorescence intensity compared to controls in the absence of HP-1 and HP-2. The data show very little inhibitory effect of HP-1 and HP-2 on the CYP450 enzymes.

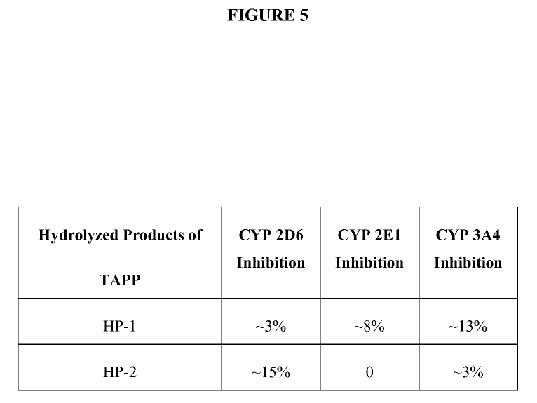

FIG. 6. Development of an in vitro model of light-mediated oxidative stress. An in vitro model of oxidative stress was developed to determine the therapeutic potency of the TAPP compositions. In this model, explants of wild-type mouse retina are placed into culture media and exposed to intense white light over a 2 day period. Control samples were exposed to subdued lighting (˜100 Lux). The formation of oxidized arachadonic acid (isoprostanes), which is a direct measure of oxidative stress in biological samples, was monitored with a commercially available assay kit (Cayman Chemical Company, Ann Arbor, Mich.). The data show that after 2 days of intense light treatment (˜10,000 Lux), retinal explants (from 2.5 month old mice) released significantly more isoprostane into the culture medium versus control samples.

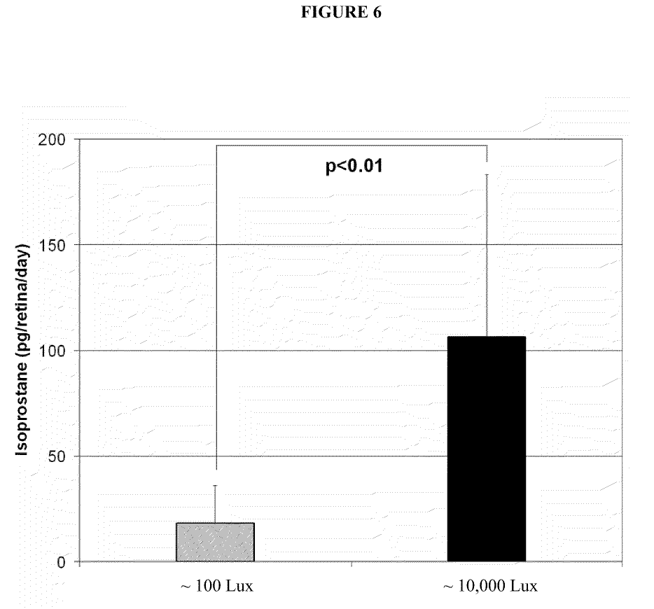

FIG. 7. TAPP1 reduces light-mediated oxidative stress. The aforementioned in vitro assay of light-mediated oxidative stress was employed to determine the therapeutic potency of TAPP1. In this experiment, isoprostane released from retina explants was measured in the absence and presence of TAPP1 in subdued lighting (˜100 Lux) and during intense light exposure (˜10,000 Lux). Following the 2-day incubation period, isoprostane release by retinas which were exposed to intense light was several-fold higher than in retinas which were maintained in subdued lighting. The presence of TAPP1 significantly reduced isoprostane release in the retinas exposed to intense light. The data indicate that TAPP1 possesses a profound anti-oxidant activity.

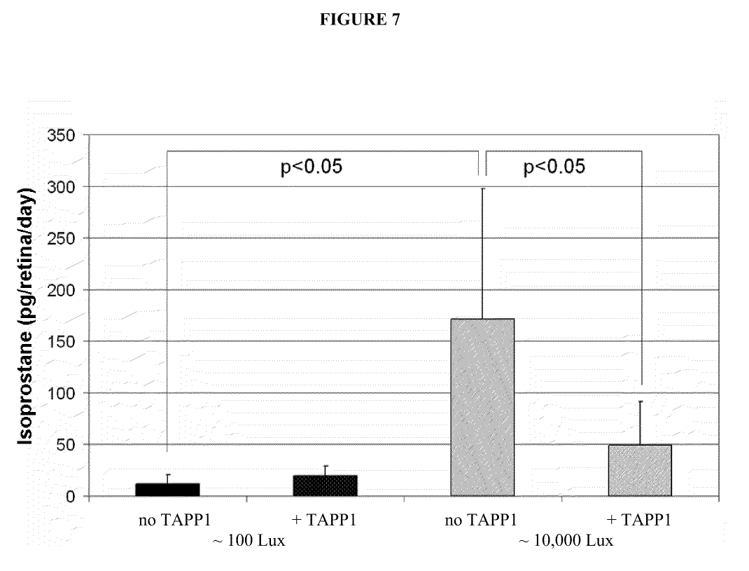

FIG. 8. Measurement of oxidative stress in the superoxide dismutase 1-(SOD1) deficient mouse. SOD1 is the most highly active and abundant free radical scavenger in the mammalian retina. The SOD1-deficient mouse manifests a phenotype which is consistent with age-related macular degeneration (i.e., formation of drusen, thickening of Bruch's membrane and choroidal neovascularization). We sought to determine whether the SOD1-deficient mouse would be an appropriate animal model to evaluate the therapeutic efficacy of the TAPP compositions in vivo. Under conditions of oxidative stress, isoprostane species are excreted into the urine. Thus, a study was performed to determine whether steady-state isoprostane levels were increased in the SOD1 mutant mice compared to age and strain matched wild-type mice. The data reveal a ˜4-fold increase in urine isoprostane levels in the SOD1 mutant mice.

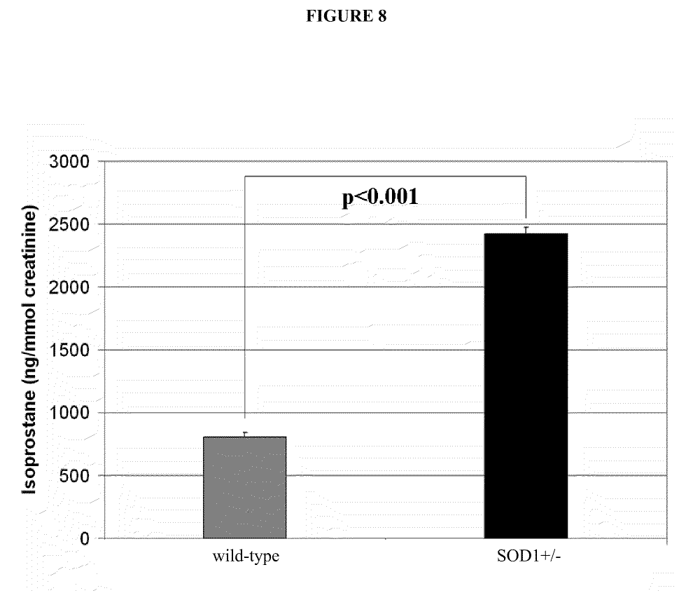

FIG. 9. TAPP1 reduces oxidative stress in SOD1 mutant mice. The evolution of oxidative stress and retinal pathology in SOD1 mutant mice, is preceded by the formation of lipid hydroperoxides (LH), and conjugates of malondialdehyde (MDA) and nitrotyrosine (NT). These conjugates are detected in situ using appropriate monoclonal antibodies. To determine the therapeutic efficacy of the TAPP compositions in vivo, we probed fixed sections of eye tissues for these biomarkers following treatment with either TAPP1 (eye drop formulation containing 40 mM TAPP1 in 45% β-cyclodextrin) or the TAPP1 vehicle (β-cyclodextrin alone). Mice (aged 3 months) were treated with one drop per day, 5 treatments per week for 6 weeks, for a total of 30 treatments. In these studies, areas of increased light intensity indicate increased amounts of LH, MDA or NT in the tissue sections. The data show that in ocular tissues of mice treated with the TAPP1 vehicle, there is pronounced LH, MDA and NT immunoreactivity (panels A, C and E, respectively). In contrast, mice treated with TAPP1 showed significantly reduced LH, MDA and NT immunoreactivity (panels B, D and F, respectively).

FIG. 10. TAPP1 improves integrity of retinal vessels in SOD1 mutant mice. A late stage pathology which has been documented in SOD1 mutant mice is choroidal neovascularization (CNV). This pathology is detected by fluorescein angiography. In this technique, a fluorescent dye (fluorescein) is injected into mice and the flow of this dye through vessels of the retina is monitored using a specialized fundus camera. Leakage of the dye outside the retinal vessels indicates compromised vessel integrity and/or CNV. SOD1 mutant mice (aged 6 months) which have been administered the TAPP1 vehicle (treatment protocol is described above) show a prominent area of dye leakage in the area surrounding the optic disc (indicated by arrow). However, there is no dye leakage in mice treated with TAPP1. These data indicate that TAPP1 has a therapeutic effect on improving the integrity of retinal vessels.

FIG. 11. Purity of [¹⁴C] Compound 14 determined by HPLC. [¹⁴C]Compound 14 was prepared as described in The Examples. It is prepared as a 23 mM solution in ethanol with specific activity of 52 μCi/μM. A 30 μl aliquot of a 30-fold diluted sample (in 10% acetonitrile) was analyzed by HPLC using a liquid chromatograph (Agilent 1100 Series; Agilent Technologies, Palo Alto, Calif.) equipped with a diode-array detector and connected in line with a radiometric flow scintillation analyzer (FSA, Perkin Elmer, Waltham, Mass.). The sample was chromatographed on an Agilent Zorbax 300SB-C18 5-μm column (250×4.6-mm; Agilent Technologies) using a linear gradient over 15 minutes from a concentration of acetonitrile/water/glacial acetic acid 0:100:0.02 (v:v:v) to 70:30:0.02 (v:v:v) at a flow rate of 1 mL/min and column temperature of 40° C. Identity of the indicated compounds was confirmed by online spectral analysis and by co-elution with authentic standards. Panel A shows the absorbance chromatogram at wavelength of 220 nm, and panel B shows the radioactivity chromatogram. The majority of the radioactivity (95%) of the sample (green peak) is associated the main Compound 14 peak eluting at 11.5 min; the remaining 5% radioactivity (pink peak) comes from a minor peak eluting at 13 min, which is the oxidized form of Compound 14. Other peaks in absorbance chromatogram are due to solvent front (two peaks around 5 min) and very low quantity of impurities, which have no associated radioactivity. The results show the [¹⁴C]Compound 14 preparation has reasonably good purity and specific activity.

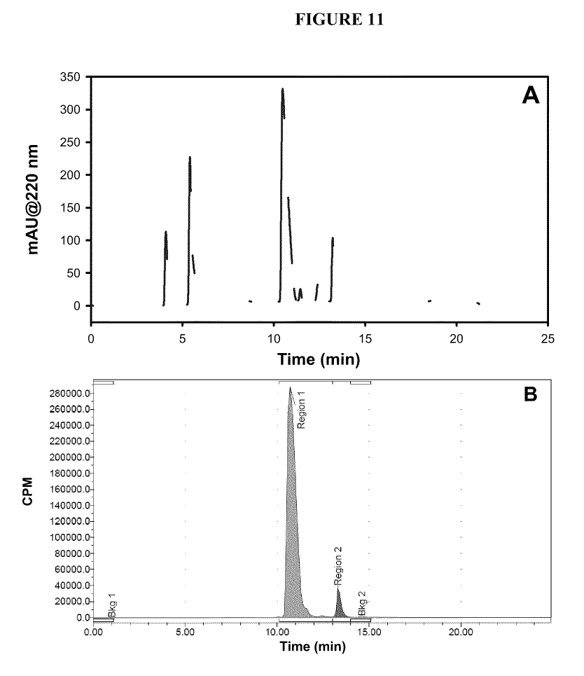

FIG. 12. Dose-response of in vitro anti-oxidation activity of [¹⁴C]Compound 14. The anti-oxidation assay measures the potency of the test compound to inhibit the metmyoglobin-mediated oxidation of ABTS to ABTS^{•+} radical cation. [¹⁴C]Compound 14 (0, 5, 10, 20, 50, 100, 200, and 500 μM) was mixed with a solution containing 15 μM ABTS and 2.5 μM metmyoglobin at room temperature. Hydrogen peroxide at 80 μM was then added to the mixture to activate metmyoglobin to ferrylmyoglobin radical, which in turn oxidizes ABTS to ABTS^{•+}. The oxidized ABTS produced a green color. Anti-oxidant potency was calculated from the inhibition of ABTS^{•+} formation, based on the sample absorbance at 750 nm. [¹⁴C]Compound 14 demonstrated a dose-dependent anti-oxidation activity, with IC50 value of about 10 μM. As a reference, the anti-oxidation activity of non-radioactive Compound 14 is shown (solid trace).

FIG. 13. Pharmacokinetics and ocular tissue distribution of total radioactivity following single topical dose administration of [¹⁴C]Compound 14 in mice. Seven ABCA4+/−/SOD+/− mice were used for this study. The mice were anesthetized and drug was administered as described above. At each specified time post dosing (0 min, 15 min, 30 min, 1 hr, 2 hr, 4 hr and 6 hr), one mouse was sacrificed by cervical dislocation and both eyeballs were enucleated. Each eyeball was rinsed in 1 ml fresh PBS to wash off unabsorbed drug. The right eye was analyzed intact. From the left eye, the following tissues were harvested: lens, anterior segment, retina and posterior segment. Total radioactivity of all tissues was determined using a liquid scintillation analyzer. The data show that [¹⁴C]Compound 14 is taken into ocular tissues within 15 min post treatment. Peak drug concentration in all tissues occurred at ˜1 hr. The highest concentrations of [¹⁴C]Compound 14 were found in the anterior and posterior segments. Lower concentrations of [¹⁴C]Compound 14 were observed in the lens and retina. The pattern and time course for drug clearance from these tissues was also comparable.

FIG. 14. Pharmacokinetics and ocular tissue distribution of total radioactivity following single topical dose administration of [¹⁴C]Compound 14 in rabbit. Four New Zealand White rabbits, age 3-4 months, weight 2.7 to 3.2 kg were used for this study. The rabbits were anesthetized and drug was administered as described above. At each specified time post dosing (0.5, 1, 2, and 4 hr), blood was collected from the ear vein of one rabbit and recovered serum volume was recorded. Rabbits were sacrificed by asphyxiation and both eyeballs were enucleated. Each eyeball was rinsed in 10 ml fresh PBS to wash off unabsorbed drug. The following tissues were harvested from each eye: anterior segment, lens, vitreous body, retina and posterior segment (without retina). Total radioactivity of each tissue and in serum was determined using a liquid scintillation analyzer (Packard Tri-Carb 2100TR). CPM was converted to DPM, and quantity of the drug (pmole) absorbed in each tissue was calculated based on specific activity (average DPM in tissues from left and right eyes). The serum level of the drug was determined as concentration (pmole/ml). [¹⁴C]Compound 14 was rapidly taken into ocular tissues (within ˜15 min) and peak drug concentration in all tissues occurred at ˜0.5 hr. The highest recovery of [¹⁴C]Compound 14 was observed in the anterior and posterior segments. Recovery of [¹⁴C]Compound 14 was approximately 10-fold lower in retina and lens, while the vitreous body contained an intermediate amount of [¹⁴C]Compound 14. The pattern and time course for drug clearance from these tissues was comparable.

### DETAILED DESCRIPTION OF THE INVENTION

The methods, compounds, and compositions described herein find use in the treatment of ophthalmic conditions characterized by oxidative stress or damage. In one aspect, the ophthalmic condition characterized by oxidative stress or damage is a vitreoretinal disease or condition. In other aspects, the ophthalmic condition is diabetic retinopathy or age-related macular degenerations.

Oxidative damage plays a role in the pathogenesis of many diseases such as, for example, age-related diseases. For example, UV exposure generates free radicals and ROS detrimental to cells and tissues. Free radicals and ROS have the potential to damage cells and tissues in the eye. ROS are toxic and can cause lipid peroxidation, protein oxidation and mutagenesis. Lipid peroxidation occurs in response to elevated levels of ROS with the liberation of reactive aldehydes, such as malondialdehyde (MDA). Accumulation of ROS-induced oxidative damage contributes to age-related eye diseases such as macular degeneration, glaucoma, cataracts, and other eye diseases. Diabetes, smoking, exposure to excessive sunlight, and ozone contribute to oxidative stress.

Free radical scavengers and antioxidants play a role in the prevention and treatment of diseases caused by oxidative stress. Nitroxides, also known as aminoxyl radicals are free radicals derived from hydroxylamines by removal of the hydrogen atom from the hydroxy group, have radical scavenging properties by inhibiting the reaction of superoxide and nitric oxide to produce peroxinitrite. Nitroxides possess antioxidant and protective capabilities that are beneficial to conditions where free radicals and ROS are implicated.

Not wishing to be bound by theory, administration of at least one nitroxide compound that reduces the reactive oxygen species in the eye of a mammal is used to treat ophthalmic conditions characterized by oxidative stress or damage.

Conditions characterized by oxidative damage include any condition of the eye where oxidative stress and/or damage causes or contributes to the onset of the condition. Various cell types in the eye are susceptible to both photochemical and non-photochemical damage caused by oxidative stress and/or damage. For example, the lens is susceptible to oxidative damage. When exposed to the action of exogenous and endogenous ROS, crystalline proteins in the lens may cross-link and aggregate. The retina is also susceptible to oxidative damage. Long-term exposure to radiation can damage photoreceptor outer segments, inhibit mitosis in the retinal pigment epithelium and choroids, and may cause photoreceptor degeneration and lipid peroxidation. Further, polyunsaturated fatty acids found in the lens and in the photoreceptor membranes of the rods and cones of the retina are susceptible to oxidative damage. Lipid radicals caused by oxidation may result in losses in function and structural integrity, such as loss of retinal cells, accumulation of lipofuscin within the retinal pigment epithelium, drusen formation, accumulation of degraded products in Bruch's membrane and changes in choroidal capillaries. The cornea is also susceptible to oxidative stress because, for example, the comea is exposed to a wide spectrum of light. Reactive oxygen species cause oxidative damage to cytoplasmic and nuclear elements of cells and cause changes to the extracellular matrix. Accumulation of oxidative damage throughout life is believed to be a major contributory factor in tissue aging.

Examples of such conditions characterized by oxidative damage include, but are in no way limited to, diabetic retinopathy, age-related macular degeneration, macular edema, glaucoma, ocular hypertension, cataracts, optic neuropathy, keratoconus and bullous keratophaty and Fuchs' endothelial dystrophy.

The term “ophthalmic disease or condition” refers to any disease or condition involving the eye or related tissues. Non-limiting examples include diseases or conditions involving degeneration of the retina and/or macula, such as the retinal and/or macular dystrophies and the retinal and/or macular degenerations, and corneal disorders, such as keratoconus and bullous keratophaty and Fuchs' endothelial dystrophy.

The term “vitreoretinal disease” refers to any disease or condition involving the vitreous and retina, such as, by way of example only, diabetic retinopathy, macular degeneration, vitreoretinopathy, endopthalmitis, retinopathy of prematurity, retinal vascular diseases, macular edema, AIDS-related retinitis, posterior segment uveitis, and retinitis pigmentosa.

### Diabetic Retinopathy

Diabetic retinopathy is retinopathy caused by complications of diabetes mellitus, both Type I and Type II. Diabetic retinopathy is an ocular manifestation of systemic disease which usually affects diabetics who have had the disease for several years. Small blood vessels in the eye are vulnerable to poor blood sugar control, and high blood sugar can damage these blood vessels, causing them to leak fluid or bleed and causing the retina to swell and form deposits. In a later stage, new blood vessels may grow on the surface of the retina, leading to serious vision loss and retinal scarring, and may eventually lead to blindness. Symptoms of diabetic retinopathy may include vision loss or blurred vision, dark or empty spots in the center of vision, poor night vision, and/or difficulty adjusting from bright light to dim light.

Oxidative stress has been implicated in the pathogenesis of diabetic retinopathy. Not wishing to be bound by theory, it has been hypothesized that hyperglycemia damages the retina and vascular epithelium by inducing the synthesis of ROS. Levels of antioxidants such as glutathione and superoxide dismutase (SOD) are decreased in retinopathic conditions due to higher lipid peroxidation. The concentration of lipid peroxidation product as measured by concentration of malondialdehyde (MDA) and 4-hydroxynonenal in patients with retinopathy was found to be elevated in comparison to diabetic patients without retinopathy and healthy patients. Polak M and Zagorski Z, Ann Univ Maria Curie Sklodowska [Med] 59(1):434-7 (2004).

### Age-Related Macular Degeneration

Macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type. In dry macular degeneration, the deterioration of the retina is associated with the formation of small yellow deposits, known as drusen, under the macula; in addition, the accumulation of lipofuscin in the RPE leads to geographic atrophy. This phenomena leads to a thinning and dying out of the macula. The location and amount of thinning in the retina caused by the drusen directly correlates to the amount of central vision loss. Degeneration of the pigmented layer of the retina and photoreceptors overlying drusen become atrophic and can cause a slow loss of central vision.

In “wet” macular degeneration new blood vessels form (i.e., neovascularization) to improve the blood supply to retinal tissue, specifically beneath the macula, a portion of the retina that is responsible for our sharp central vision. The new vessels are easily damaged and sometimes rupture, causing bleeding and injury to the surrounding tissue. Although wet macular degeneration only occurs in about 10 percent of all macular degeneration cases, it accounts for approximately 90% of macular degeneration-related blindness. Neovascularization can lead to rapid loss of vision and eventual scarring of the retinal tissues and bleeding in the eye. This scar tissue and blood produces a dark, distorted area in the vision, often rendering the eye legally blind. Wet macular degeneration usually starts with distortion in the central field of vision. Straight lines become wavy. Many people with macular degeneration also report having blurred vision and blank spots in their visual field. Growth promoting proteins called vascular endothelial growth factor, or VEGF, have been targeted for triggering this abnormal vessel growth in the eye. Studies have shown that anti-VEGF agents can be used to block and prevent abnormal blood vessel growth. Such anti-VEGF agents stop or inhibit aberrant growth of endothelial tissues, so there is less growth of blood vessels. Such anti-VEGF agents are successful in anti-angiogenesis or blocking VEGF's ability to induce blood vessel growth beneath the retina, as well as blood vessel leakiness.

Oxidative stress has been implicated in the pathogenesis of age-related macular degeneration. During aging, damage to macromolecules such as membrane phospholipids within the eye has been proposed to lead to macular degeneration. High polyunsaturated fatty acid content of photoreceptor membranes particularly expose the retina to increased risk of lipid peroxidation by unopposed action of free radicals. Not wishing to be bound by theory, it is suggested that the retina is susceptible to lipid peroxidation and that this susceptibility increases with aging in the macular region. Increased lipid peroxidation in serum samples from age-related macular degeneration patients, as measured by plasma level of MDA, was consistent with the role of oxidative stress in the disease. Totan Y et al., Br J. Opthalmol 85:1426-28 (2001).

### Stargardt's Disease

Stargardt's Disease is a macular dystrophy that is inherited as an autosomal recessive disorder, with an onset during childhood. See, e.g., Allikmets et al., Science, 277:1805-07 (1997); Lewis et al., Am. J. Hum. Gen., 64:422-34 (1999): Stone et al., Nature Gen., 20:328-29 (1998); Allikmets, Am. J. Hum. Gen., 67:793-799 (2000); Klevering et al., Opthalmology, 111:546-553 (2004).

Stargardt's Disease is characterized clinically by progressive loss of central vision and progressive atrophy of the RPE overlying the macula. Mutations in the human ABCA4 gene for Rim Protein (RmP) are responsible for Stargardt's Disease. Early in the disease course, patients show delayed dark adaptation but otherwise normal cone function. Histologically, Stargardt's Disease is associated with deposition of lipofuscin pigment granules in RPE cells.

Mutations in ABCA4 have also been implicated in recessive retinitis pigmentosa, see, e.g., Cremers et al., Hum. Mol. Genet., 7:355-62 (1998), recessive cone-rod dystrophy, see id., and non-exudative age-related macular degeneration, see e.g., Allikmets et al., Science, 277:1805-07 (1997); Lewis et al., Am. J. Hum. Genet., 64:422-34 (1999), although the prevalence of ABCA4 mutations in AMD is still uncertain. See Stone et al., Nature Gen., 20:328-29 (1998); Allikmets, Am. J. Hum. Gen., 67:793-799 (2000); Klevering, et al, Opthalmology, 111:546-553 (2004). Similar to Stargardt's Disease, these diseases are associated with delayed rod dark-adaptation. See Steinmetz et al., Brit. J Ophihalm., 77:549-54 (1993). Lipofuscin deposition in RPE cells is also seen prominently in AMD, see Kliffen et al., Microsc. Res. Tech., 36:106-22 (1997) and some cases of retinitis pigmentosa. See Bergsma et al., Nature, 265:62-67 (1977).

In addition, there are several types of macular degenerations that affect children, teenagers or adults that are commonly known as early onset or juvenile macular degeneration. Many of these types are hereditary and are looked upon as macular dystrophies instead of degeneration. Some examples of macular dystrophies include: Cone-Rod Dystrophy, Corneal Dystrophy, Fuch's Dystrophy, Sorsby's Macular Dystrophy, Best Disease, and Juvenile Retinoschisis, as well as Stargardt's Disease.

### Glaucoma

Glaucoma is a disease of the optic nerve involving loss of retinal ganglion cells in a characteristic pattern of optic neuropathy. It is a disorder associated with pressure in the eye and is characterized by damage to the optic nerve with consequent visual loss, initially peripheral, but potentially blinding. Although raised intraocular pressure is a significant risk factor for developing glaucoma, there is no set threshold for intraocular pressure that causes glaucoma. Eye pressure, perfusion of the optic nerve, mechanical factors in and around the optic nerve, and biochemical factors may also play a role in the pathogenesis of glaucoma. Primary open angle glaucoma (POAG) is the most common of all types of glaucoma. The condition is diagnosed in the presence of an open angle, evidence of optic nerve damage, and peripheral vision loss consistent with glaucoma on a visual field test.

Risk factors for glaucoma include elevated intraocular pressure, family history of glaucoma, advanced age, cardiovascular disease, diabetes mellitus, myopia, and high blood pressure, to name a few. Oxidative damage and lipid peroxidation have also been found to have a role in the pathogenesis of POAG, as measured by elevated levels of plasma MDA in patients with POAG. Yildirim O, Eye 19(5):580-3 (2005).

Other factors that may contribute to conditions of the eye caused by oxidative stress or damage can be further caused or exacerbated by, e.g., diabetes, hypertension, arteriosclerosis, macular drusen, or smoking of tobacco.

### Compounds of Formulas I-V

The compounds of Formulas I, II, IIIa, IIIb, IV and V contain N-oxyl moieties, and are useful for the treatment of ophthalmic conditions characterized or caused by oxidative stress or damage.

In one aspect are compounds of Formula I or a pharmaceutically acceptable solvate or a pharmaceutically acceptable salt thereof:

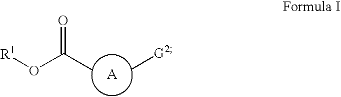

wherein,
R¹ is N-oxyl-2,2,6,6-tetramethylpiperidin-4-yl or N-oxyl-2,2,5,5-tetramethylpyrrolidin-3-yl;

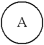

is an optionally substituted 5-membered heterocycle containing at least 1 N atom in the heterocyclic ring, or an optionally substituted 6-membered heterocycle containing at least 1 N atom in the heterocyclic ring; and
G² is selected from H, C₁-C₆ alkyl, —CF₃, —CN, —CO₂H, —CO₂R², tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄alkyl group or an optionally substituted phenyl group;
each R³ is independently selected from H, an optionally substituted C₁-C₄ alkyl group, an optionally substituted aryl, and an optionally substituted heteroaryl.

In a further embodiment, R¹ is N-oxyl-2,2,6,6-tetramethylpiperidin-4-yl. In a further or alternative embodiment,

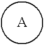

is an optionally substituted 5-membered heteroaryl containing at least 1 N atom in the heteroaryl ring or an optionally substituted 6-membered heteroaryl containing at least 1 N atom in the heteroaryl ring. In yet a further or alternative embodiment, G² is H, methyl, ethyl, CF₃, CN, CO₂H, CO₂Me, CO₂Et, tetrazolyl, —NHS(═O)₂Me, —NHS(═O)₂Ph, —S(═O)₂NH₂, S(═O)₂NHMe, OH, —OMe, —C(═O)CF₃, —C(O)NHS(═O)₂Me, —S(═O)₂NHC(═O)Me, optionally substituted aryl, or an optionally substituted heteroaryl. In still a further or alternative embodiment,

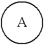

is an optionally substituted group selected from pyrazolylene, isoxazolylene, isothiazolylene, pyrrolylene, oxazolylene, thiazolylene, imidazolylene, pyridinylene, pyrimidinylene and pyrazinylene. In still a further or alternative embodiment, G² is selected from an optionally substituted aryl and an optionally substituted heteroaryl. In still a further or alternative embodiment, G² is selected from an optionally substituted phenyl and an optionally substituted heteroaryl containing at least 1 N atom in the heteroaryl ring. In still a further or alternative embodiment,

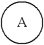

is an optionally substituted group selected from pyrazolylene, isoxazolylene, isothiazolylene, pyrrolylene, oxazolylene, thiazolylene, and imidazolylene. In still a further or alternative embodiment, G² is an optionally substituted group selected from phenyl, pyrazolyl, imidazolyl, pyridinyl, pyrimidinyl and pyrazinyl. In still a further or alternative embodiment,

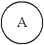

is an optionally substituted group selected from pyrazolylene, isoxazolylene, and isothiazolylene. In still a further or alternative embodiment, G² is an optionally substituted phenyl or pyridinyl.

In a further or alternative embodiment, the compound of Formula I has the structure of Formula II:

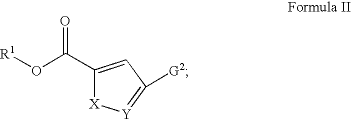

wherein;
X is O, S, NH or CH₂;
Y is CH or N;
R¹ is N-oxyl-2,2,6,6-tetramethylpiperidin-4-yl;
G² is a (substituted or unsubstituted aryl) or a (substituted or unsubstituted heteroaryl).

In still a further or alternative embodiment, Y is N. In still a further or alternative embodiment, X is O, S, or NH. In still a further or alternative embodiment, X is NH. In still a further or alternative embodiment, G² is a (substituted or unsubstituted phenyl) or a (substituted or unsubstituted heteroaryl containing at least 1 N atom in the heteroaryl ring). In still a further or alternative embodiment, G² is a (substituted or unsubstituted phenyl), (substituted or unsubstituted 5-membered heteroaryl containing at least 1 N atom in the heteroaryl ring), or a (6-membered heteroaryl containing at least 1 N atom in the heteroaryl ring). In still a further or alternative embodiment, G² is a substituted or unsubstituted group selected from phenyl, pyrazolyl, imidazolyl, pyridinyl, pyrimidinyl and pyrazinyl. In still a further or alternative embodiment, G² is a (substituted or unsubstituted phenyl) or a (6-membered heteroaryl containing at least 1 N atom in the heteroaryl ring). In still a further or alternative embodiment, G² is a substituted or unsubstituted group selected from phenyl, pyridinyl, pyrimidinyl and pyrazinyl.

In further or alternative embodiment, the compound is selected from:
N-oxyl-2,2,6,6-tetramethylpiperidin-4-yl 3-(phenyl)-1H-pyrazole-5-carboxylate;
N-oxyl-2,2,6,6-tetramethylpiperidin-4-yl 3-(pyridin-4-yl)-1H-pyrazole-5-carboxylate;
N-oxyl-2,2,6,6-tetramethylpiperidin-4-yl isonicotinate;
N-oxyl-2,2,6,6-tetramethylpiperidin-4-yl 5-methylpyrazine-2-carboxylate;
N-oxyl-2,2,6,6-tetramethylpiperidin-4-yl picolinate; and
N-oxyl-2,2,6,6-tetramethylpiperidin-4-yl nicotinate.

In one embodiment, the compound of Formula II has the formula:

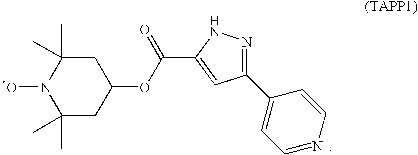

In another aspect are compounds of Formula IIIa or a pharmaceutically acceptable solvate, or a pharmaceutically acceptable salt thereof:

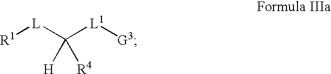

wherein,
R¹ is N-oxyl-2,2,6,6-tetramethylpiperidin-4-yl, N-oxyl-2,2,6,6-tetramethylpiperidin-4-oximyl or N-oxyl-2,2,5,5-tetramethylpyrrolidin-3-yl;
L is —OC(═O)—, —C(═O)O—, —OCH₂— or —CH₂O—;
L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl.

In a further or alternative embodiment, R¹ is N-oxyl-2,2,6,6-tetramethylpiperidin-4-yl. In still a further or alternative embodiment, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted aryl, and an optionally substituted heteroaryl. In still a further or alternative embodiment, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted phenyl, optionally substituted pyrazolyl, optionally substituted imidazolyl, optionally substituted pyridinyl, optionally substituted pyrimidinyl, and optionally substituted pyrazinyl. In still a further or alternative embodiment, G³ is selected from —CO₂H, —CO₂R², tetrazolyl, optionally substituted aryl, and an optionally substituted heteroaryl. In still a further or alternative embodiment, R⁴ is H. In still a further or alternative embodiment, L¹ is an optionally substituted C₁-C₈alkylene optionally containing at least one unit of unsaturation. In still a further or alternative embodiment, L¹ is a bond, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH═CH—, or —CH₂CH₂CH₂CH₂CH(OH)CH═CH—. In still a further or alternative embodiment, L is —OC(═O)— or —OCH₂—. In still a further or alternative embodiment, G³ is selected from —CO₂H, —CO₂R² and tetrazolyl. In still a further or alternative embodiment, R⁴ is N(R⁵)₂; and R⁵ is H. In still a further or alternative embodiment, -L¹-G³ is selected from H, —CH₃, —CH₂CH(CH₃)₂, CH₂CO₂H, —CH₂CH₂CO₂H, —CH₂CH₂CH₂CH═CHCO₂H, —CH₂CH₂CH₂CH₂CH(OH)CH═CHCO₂H, —CH₂CONH₂, —CH₂CH₂CONH₂, —CH₂CH₂CH₂CH₂NH₂, —CH₂CH₂CH₂NC(═NH)NH₂, —CH₂OH, —CH₂CH₂SCH₃, —CH(OH)CH₃, —CH₂SH, —CH(CH₃)₂, —CH(CH₃)CH₂CH₃, —CH₂-imidazolyl, —CH₂-(1H-indol-3-yl), —CH₂-phenyl, —CH₂-(4-hydroxyphenyl). In still a further or alternative embodiment, L is —OC(═O)—.

In a further or alternative embodiment is a compound selected from:
1-N-oxyl-2,2,6,6-tetramethylpiperidin-4-yl 2-(pyridin-2-yl)acetate;
1-N-oxyl-2,2,6,6-tetramethylpiperidin-4-yl 2-amino-3-phenylpropanoate;
4-(1-N-oxyl-2,2,6,6-tetramethylpiperidin-4-yloxy)-4-yl succinate; and
(E)-9-((N-oxyl-2,2,6,6-tetramethylpiperidin-4-yloxy)carbonyl)-4-hydroxynon-2-enoic acid.

In one embodiment, the compound of Formula IIIa has the structure:

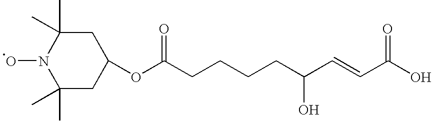

In another aspect, are compounds of Formula IIIb or pharmaceutically acceptable solvate, pharmaceutically acceptable salt, or pharmaceutically acceptable prodrug thereof

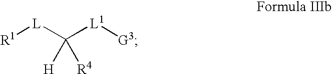

wherein,
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —NR⁶C(═O)—, —C(═O)NR⁶—, —NR⁶CH₂—, CH₂NR⁶—, or an optionally substituted C₁-C₈alkylene;

L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl;
each R⁶ is independently selected from H, an optionally substituted C₁-C₄ alkyl group, —C(═O)R², and —S(═O)₂N(R³)₂.

In some embodiments, the compound of Formula IIIb is a compound wherein R¹ is N-oxyl-2,2,6,6-tetramethylpiperidin-4-yl. In some embodiments, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(—NR³)NR³, optionally substituted aryl, and an optionally substituted heteroaryl. In other embodiments, G³ is selected from —CN, —CO₂H, —CO₂R², —C(═O)N(R³)₂, —C(═O)R², —N(R³)₂, tetrazolyl, —OH, —OR², —C(═O)CF₃, —SR³, —NR³C(═NR³)NR³, optionally substituted phenyl, optionally substituted pyrazolyl, optionally substituted imidazolyl, optionally substituted pyridinyl, optionally substituted pyrimidinyl, and optionally substituted pyrazinyl. In still further embodiments, G³ is selected from —CO₂H, —CO₂R², tetrazolyl, optionally substituted aryl, and an optionally substituted heteroaryl.

In some embodiments, the compound of Formula IIIb is a compound wherein R⁴ is H. In some embodiments of Formula (IIIb), L¹ is an optionally substituted C₁-C₈alkylene optionally containing at least one unit of unsaturation. In some embodiments, L¹ is a bond, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH═CH—, or —CH₂CH₂CH₂CH₂CH(OH)CH═CH—.

In some embodiments of Formula IIIb, L is —NR⁶C(═O)— or —NR⁶CH₂— or an optionally substituted C₁-C₈alkylene

In some embodiments of Formula IIb, G³ is selected from —CO₂H, —CO₂R² and tetrazolyl. In some embodiments, R⁴ is N(R⁵)₂; and R⁵ is H.

In some embodiments of Formula IIIb, -L¹-G³ is selected from H, —CH₃, —CH₂CH(CH₃)₂, —CH₂CO₂H, —CH₂CH₂CO₂H, —CH₂CH₂CH₂CH═CHCO₂H, —CH₂CH₂CH₂CH₂CH(OH)CH═CHCO₂H, —CH₂CONH₂, —CH₂CH₂CONH₂, —CH₂CH₂CH₂CH₂NH₂, —CH₂CH₂CH₂NC(═NH)NH₂, —CH₂OH, —CH₂CH₂SCH₃, —CH(OH)CH₃, —CH₂SH, —CH(CH₃)₂, —CH(CH₃)CH₂CH₃, —CH₂-imidazolyl, —CH₂-(1H-indol-3-yl), —CH₂-phenyl, —CH₂-(4-hydroxyphenyl).

In some embodiments of Formula IIIb, L is —NR⁶C(═O)—.

In some embodiments, the compound of Formula IIIb is (E)-9-((2,2,6,6-tetramethylpiperidin-1-oxyl)-4-aminyl)-9-oxo-4-hydroxynon-2-enoic acid; (E)-9-((2,2,6,6-tetramethylpiperidin-1-hydroxide)-4-aminyl)-9-oxo-4-hydroxynon-2-enoic acid, (E)-9-((2,2,6,6-tetramethylpiperidin-1-oxyl)-4-amino-(N-acetyl))-4-hydroxynon-2-enoic acid.

In one aspect is a compound having the structure of Formula IV or V:

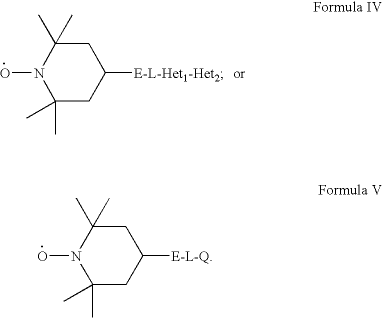

wherein E is an esterase-cleavable moiety; Het₁ and Het₂ are independently selected heterocycle moieties; L is an optionally substituted alkylene, heteroalkylene or alkenylene moiety; and Q is non-heterocyclic polar moiety.

In one embodiment is a compound having the structure of Formula IV or V, wherein E is —O—C(O)— or —C(O)—O—.

In another embodiment is a compound having the structure of Formula IV or V, wherein E is —O—C(O)— or —C(O)—O— and wherein L is an optionally substituted alkylene moiety.

In another embodiment is a compound having the structure of Formula IV or V, wherein E is —O—C(O)— or —C(O)—O— wherein L is an optionally substituted alkylene moiety, and wherein one of Het1 or Het2 is an aromatic N-containing heterocycle.

Certain compounds presented herein possess one or more stereocenters and each center exists in the R or S configuration. The compounds presented herein include all diastereomeric, enantiomeric, and epimeric forms as well as the appropriate mixtures thereof. Stereoisomers are obtained, if desired, by methods such as the separation of stereoisomers by chiral chromatographic columns.

The methods and formulations described herein include the use of N-oxides, crystalline forms (also known as polymorphs), or pharmaceutically acceptable salts of compounds having the structure of Formula I, II, IIIa, IIIb, IV or V, as well as active metabolites of these compounds having the same type of activity. In some situations, compounds exist as tautomers. All tautomers are included within the scope of the compounds presented herein. In addition, the compounds described herein exist in unsolvated as well as solvated forms with pharmaceutically acceptable solvents such as water, ethanol, and the like. The solvated forms of the compounds presented herein are also considered to be disclosed herein.

### Synthesis of the Compounds of Formula I, II, IIIa, IIIb, IV or V

Compounds described herein (e.g. compounds of Formula I, II, IIIa, IIIb, IV or V), are synthesized using standard synthetic techniques or using known methods in combination with methods described herein. In additions, solvents, temperatures and other reaction conditions presented herein are optionally varied.

A non-limiting example of a synthetic approach toward compounds of Formula I, II, IIIa, IIIb, IV or V is outlined in Scheme I.

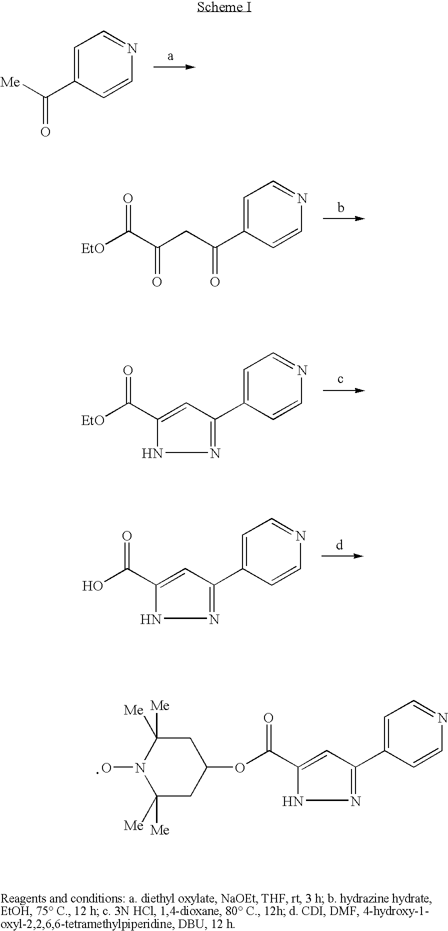

The synthesis begins with a Claisen condensation between 4-acetylpyridine and ethyl oxalate to afford the diketone. Condensation of the diketone with hydrazine hydrate provides the substituted pyrazole. Hydrolysis of the ester followed by coupling of the resulting acid to 4-hydroxy-1-oxyl-2,2,6,6-tetramethylpiperidine yields the desired product 1-oxyl-2,2,6,6-tetramethylpiperidin-4-yl-3-(pyridin-4-yl)-1H-pyrazole-5-carboxylate. Additional compounds of Formula I, II, IIIa, IIIb, IV or V are prepared using the methodology outlined above and in the Examples.

The starting material used for the synthesis of the compounds described herein are synthesized or are obtained from commercial sources, such as, but not limited to, Aldrich Chemical Co. (Milwaukee, Wis.), or Sigma Chemical Co. (St. Louis, Mo.). The compounds described herein, and other related compounds having different substituents are synthesized using techniques and materials, such as described, for example, in March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). The reactions are optionally modified by the use of appropriate reagents and conditions for the introduction of the various moieties found in the formulae as provided herein. As a guide the following synthetic methods are optionally utilized.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof” Precursor functional groups are shown as electrophilic groups and nucleophilic groups. The functional group on the organic substance is optionally attached directly, or attached via any useful spacer or linker as defined below.

TABLE 2
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are optionally blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties are also optionally blocked with hydrolytically removable protective groups such as the benzyl group, while amine groups capable of hydrogen bonding with acids are optionally blocked with base labile groups such as Fmoc. Carboxylic acid reactive moieties are optionally protected by conversion to simple ester derivatives as exemplified herein, or they are optionally blocked with oxidatively-removable protective groups such as 2,4-dimethoxybenzyl, while co-existing amino groups are optionally blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate is optionally attached. As long as the residue is attached to the resin, that functional group is blocked and cannot react. Once released from the resin, the functional group is available to react.

Examples of protecting groups are described in Greene and Wuts, Protective Groups in Organic Synthesis, 3rd Ed., John Wiley & Sons, New York, N.Y., 1999, which is incorporated herein by reference for such disclosure.

### Further Forms of Compounds

In one aspect, compounds of Formula I, II, IIIa, IIIb, IV or V are prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In another aspect, compounds of Formula I, II, IIIa, IIIb, IV or V are prepared as pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

In one aspect, compounds of Formula I, II, IIIa, IIIb, IV or V are prepared as pharmaceutically acceptable salts formed when an acidic proton present in the parent compound either is replaced by a metal ion, for example an alkali metal ion, an alkaline earth ion, or an aluminum ion; or coordinates with an organic base.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are formed during the process of isolation from the reaction mixture or crystallization with pharmaceutically acceptable solvents such as water, ethanol, and the like. Hydrates are formed when the solvent is water, or alcoholates are formed when the solvent is alcohol. Solvates of compounds of Formula I, II, IIIa, IIIb, IV or V are conveniently prepared or formed during the processes described herein. By way of example only, hydrates of compounds of Formula I, II, IIIa, IIIb, IV or V are conveniently prepared by isolation from the reaction mixture or recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

In one aspect, compounds of Formula I, II, IIIa, IIIb, IV or V are prepared or isolated in various forms, including but not limited to, amorphous forms, milled forms and nano-particulate forms. In addition, compounds of Formula I, II, IIIa, IIIb, IV or V include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

### Pharmaceutical Compositions

Other aspects are pharmaceutical compositions comprising at least one compound of Formula I, II, IIIa, IIIb, IV or V as described herein and an ophthalmically acceptable excipient. Suitable routes of administration are, for example, topical on an eye, intraocular, intraorbital, intraconal, ophthalmic, retrobulbar, and periorbital. The term “pharmaceutical composition” refers to at least one compound of Formula I, II, IIIa, IIIb, IV or V as described herein and an ophthalmically acceptable excipient.

The term “ophthalmically acceptable” with respect to a formulation, composition or ingredient typically means having no persistent detrimental effect on the treated eye or the functioning thereof, or on the general health of the subject being treated. Transient effects such as minor irritation or a “stinging” sensation are common with topical ophthalmic administration of agents and consistent with the formulation, composition or ingredient in question being “ophthalmically acceptable.”

In some embodiments, the compounds described herein are administered to a human patientper se, or in pharmaceutical compositions where they are mixed with other active ingredients, as in combination therapy, or with suitable carrier(s) or excipient(s). Techniques for formulation and administration of the compounds of the instant application are found, e.g., in Remington's, The Science and Practice of Pharmacy, 20th ed. (2000).

Topical administration to an eye can be formulated as, for example, eye drops, eye ointment, eye creams, eye wash, eye solutions, suspensions, spray, lotions, gels, pastes, medicated sticks, balms, or shampoos. In one embodiment, the composition is the form of eye drops that can be applied topically on the eye of a mammal, including a human. The topical formulation of the pharmaceutical compositions provided herein in some embodiments, also comprise liposomes, micelles, microspheres, nanospheres or nanoparticles, and mixtures thereof.

For example, pharmaceutical compositions are formulated in conventional manner using one or more ophthalmically acceptable excipients which facilitate processing of the active compounds into preparations which are used pharmaceutically. Proper formulation is dependent upon the route of administration chosen.

Administration of a composition to the eye generally results in direct contact of the agents with the cornea, through which at least a portion of the administered agents pass. Often, the composition has an effective residence time in the eye of about 2 to about 24 hours, more typically about 4 to about 24 hours and most typically about 6 to about 24 hours.

A composition comprising a compound of Formula I, II, IIIa, IIIb, IV or V can illustratively take the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous. Alternatively, the composition can take the form of an ointment.

Useful compositions can be an aqueous solution, suspension or solution/suspension, which can be presented in the form of eye drops. A desired dosage can be administered via a set number of drops into the eye. For example, for a drop volume of 25 μl, administration of 1-6 drops will deliver 25-150 μl of the composition. Aqueous compositions typically contain from about 0.01% to about 50%, more typically about 0.1% to about 20%, still more typically about 0.2% to about 10%, and most typically about 0.5% to about 5%, weight/volume of active agent, such as a compound of Formula I, II, IIIa, IIIb, IV or V.

Typically, aqueous compositions have ophthalmically acceptable pH and osmolality. Useful aqueous suspension can also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions can also comprise an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions also include ophthalmically acceptable solubilizing agents to aid in the solubility of an agent, such as a compound of Formula I, II, IIIa, IIIb, IV or V. The term “solubilizing agent” generally includes agents that result in formation of a micellar solution or a true solution of the agent. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

Useful compositions also include one or more ophthalmically acceptable pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

Useful compositions also include one or more ophthalmically acceptable salts in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful compositions also include one or more ophthalmically acceptable preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more ophthalmically acceptable surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

Aqueous suspension compositions can be packaged in single-dose non-reclosable containers. Alternatively, multiple-dose reclosable containers can be used, in which case it is typical to include a preservative in the composition.

The ophthalmic composition is also optionally in the form of a solid article that can be inserted between the eye and eyelid or in the conjunctival sac, where it releases the agent. Release is to the lacrimal fluid that bathes the surface of the comea, or directly to the cornea itself, with which the solid article is generally in intimate contact. Solid articles suitable for implantation in the eye in such fashion are generally composed primarily of polymers and can be biodegradable or non-biodegradable.

Formulations for topical administration to an eye also include agents for retaining the pharmaceutical composition in the eye, for example, retaining the pharmaceutical composition at the site of action in the eye. Drug delivery to the posterior segments of the eye (e.g., to the retina, choroid, vitreous and optic nerve) is important for treating several disorders such as age-related macular degeneration, diabetic retinopathy, macular edema, uveitis, vitreoretinopathy and glaucoma. Due to anatomic membrane barriers such as the cornea, conjunctiva and sclera and lachrymal drainage, it may be difficult to achieve therapeutic drug concentrations in the posterior part of the eye from topical administration of the pharmaceutical composition.

To improve drug delivery at therapeutic concentrations to the posterior part of the eye, the pharmaceutical composition is optionally complexed with a solubilizing agent, for example, a glucan sulfate. Glucan sulfates which can be used include dextran sulfate, cyclodextrin sulfate and β-1,3-glucan sulfate, both natural and derivatives thereof, or any compound which can temporarily bind to and be retained at tissues which contain fibroblast growth factor (FGF), which improves the stability and/or solubility of a drug, and/or which improve penetration and ocular absorption of a topically administered composition in the eye. Cyclodextrin derivatives that can be used as a solubilizing agent include, for example, x-cyclodextrin, β-cyclodextrin, γ-cyclodextrin, hydroxyethyl β-cyclodextrin, hydroxypropyl γ-cyclodextrin, hydroxypropyl β-cyclodextrin, sulfated β-cyclodextrin, sulfated β-cyclodextrin, sulfobutyl ether α-cyclodextrin.

The concentration of the solubilizing agent used in the compositions and methods disclosed herein optionally vary according to the physiochemical properties, pharmacokinetic properties, side effect or adverse events, formulation considerations, or other factors associated with the therapeutic agent. The properties of other excipients in a composition may also be a factor. Thus, the concentration or amount of solubilizing agent used in accordance with the compositions and methods disclosed herein optionally vary. For example of such agents for retaining the pharmaceutical composition in the eye, see, for example, U.S. Pat. Nos. 5,227,371 and 6,969,706, which are hereby incorporated by reference for such disclosure.

Another useful formulation for administration of a compound of Formula I, II, IIIa, IIIb, IV or V employs transdermal delivery devices (“patches”). Such transdermal patches are used to provide continuous or discontinuous infusion of the compounds of the present invention in controlled amounts. The construction and use of transdermal patches for the delivery of pharmaceutical agents includes, e.g., U.S. Pat. No. 5,023,252. Such patches are constructed for continuous, pulsatile, or on demand delivery of pharmaceutical agents. Still further, transdermal delivery of the agents can be accomplished by means of iontophoretic patches and the like. Transdermal patches can provide controlled delivery of the compounds. The rate of absorption can be slowed by using rate-controlling membranes or by trapping the compound within a polymer matrix or gel. Conversely, absorption enhancers can be used to increase absorption. Formulations suitable for transdermal administration can be presented as discrete patches and can be lipophilic emulsions or buffered, aqueous solutions, dissolved and/or dispersed in a polymer or an adhesive. Transdermal patches are optionally placed over different portions of the patient's body, including over the eye.

Additional iontophoretic devices that can be used for ocular administration of a compound of Formula I, II, IIIa, IIIb, IV or V are the Eyegate applicator, created and patented by Optis France S. A., and the Ocuphor™ Ocular iontophoresis system developed by lomed, Inc.

In addition to the formulations described previously, the compounds are optionally formulated as a depot preparation. Such long acting formulations are optionally administered by implantation (for example subcutaneously, intramuscularly, intravitreally, or within the subconjunctiva). Thus, for example, the compounds are formulated with suitable polymeric or hydrophobic materials (for example as an emulsion in an acceptable oil) or ion exchange resins, or as sparingly soluble derivatives, for example, as a sparingly soluble salt.

Injectable depot forms are optionally made by forming microencapsulated matrices (also known as microencapsule matrices) of a compound of Formula I, II, IIIa, IIIb, IV or V in biodegradable polymers. Depending upon the ratio of drug to polymer and the nature of the particular polymer employed, the rate of drug release can be controlled. Depot injectable formulations are also be prepared by entrapping the drug in liposomes or microemulsions. By way of example only, posterior juxtascleral depots are used as a mode of administration of compounds of Formula I, II, IIIa, IIIb, IV or V. The sclera is a thin avascular layer, comprised of highly ordered collagen network surrounding most of vertebrate eye. Since the sclera is avascular it can be utilized as a natural storage depot from which injected material cannot rapidly removed or cleared from the eye. The formulation used for administration of the compound into the scleral layer of the eye can be any form suitable for application into the sclera by injection through a cannula with small diameter suitable for injection into the scleral layer. Examples for injectable application forms are solutions, suspensions or colloidal suspensions.

Alternatively, other delivery systems for hydrophobic pharmaceutical compounds are employed. Liposomes and emulsions are examples of delivery vehicles or carriers for hydrophobic drugs. Certain organic solvents such as N-methylpyrrolidone also are employed, although usually at the cost of greater toxicity. Additionally, the compounds are optionally delivered using a sustained-release system, such as semipermeable matrices of solid hydrophobic polymers containing therapeutic agent. Sustained-release capsules, depending on their chemical nature, release the compounds for a few weeks up to over 100 days. Depending on the chemical nature and the biological stability of therapeutic reagent, additional strategies for protein stabilization are employed.

All of the formulations described herein optionally benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

Many of the agents are optionally provided as salts with pharmaceutically compatible counter ions. Pharmaceutically compatible salts are optionally formed with many acids, including but not limited to hydrochloric, sulfuric, acetic, lactic, tartaric, malic, succinic, etc. Salts tend to be more soluble in aqueous or other protonic solvents than are the corresponding free acid or base forms.

### Methods of Treatment, Dosages and Combination Therapies

In some embodiments, the compositions containing the compound(s) described herein are administered to a subject in need (including, humans and other mammals) for prophylactic and/or therapeutic treatments.

In one aspect is a method for reducing ophthalmic reactive oxygen species in a subject, comprising administering to a subject a composition comprising a therapeutically effective amount of a compound of Formula I, II, IIIa, IIIb, IV or V, as described herein. In one embodiment, the subject is suffering from or at risk of suffering from an ophthalmic condition characterized by oxidative damage. In another embodiment, the ophthalmic condition is a vitreoretinal disease or condition. In yet another embodiment, the ophthalmic condition is diabetic retinopathy, wet age-related macular degeneration, dry age-related macular degeneration, Stargardt's disease, macular edema, glaucoma, ocular hypertension, cataracts, or optic neuropathy. In yet another embodiment, the subject is suffering from diabetes, hypertension, arteriosclerosis, exhibits macular drusen, or smokes, tobacco. In yet another embodiment, the administration is topical on an eye, intraocular, intraorbital, ophthalmic, retrobulbar, parenteral, oral, topical, intramuscular, transdermal, sublingual, intranasal, or respiratory. In yet another embodiment, the composition is administered topically to an eye. In yet another embodiment, the compound is administered as an eye drop, eye wash, or eye ointment formulation.

In one aspect is a method for treating an oxidative ophthalmic condition in a subject in need thereof, comprising administering to the subject a composition comprising a therapeutically effective amount of the compound of Formula I, II, IIIa, IIIb, IV or V, as described herein, wherein the ophthalmic condition is characterized by ophthalmic oxidative damage. In one embodiment, the ophthalmic condition is diabetic retinopathy, wet age-related macular degeneration, dry age-related macular degeneration, Stargardt's disease, macular edema, glaucoma, ocular hypertension, cataracts, or optic neuropathy. In another embodiment, the ophthalmic condition is diabetic retinopathy. In yet another embodiment, the ophthalmic condition is wet-age related macular degeneration or dry age-related macular degeneration. In yet another embodiment, the administration is topical on an eye, intraocular, intraorbital, ophthalmic, retrobulbar, parenteral, oral, topical, intramuscular, transdermal, sublingual, intranasal, or respiratory. In yet another embodiment, the composition is administered topically to an eye. In yet another embodiment, the compound is administered as an eye drop, eye wash, or eye ointment formulation.

Photooxidative damage of the eye can be caused by exposure to sunlight and/or U light, either for continuous or for brief periods of intense exposure. Photooxidative damage can also be caused by or exacerbated by factors such as diabetes, hypertension, arteriosclerosis, macular drusen, or smoking of tobacco. Photooxidative damage to the eyes may result in blurred vision, temporary or extended loss of vision, cataracts, photokeratitis (burn to the cornea), pterygium, or macular degeneration. Previous methods of protection from photooxidative damage to the eye include wearing protective headwear or eyewear, such as sunglasses with UV filters or photochromic (polarized) lenses.

In one aspect are methods for reducing ophthalmic photooxidative damage in a subject. Photooxidative damage results from oxidation caused by light or other types of radiation. For example, photooxidative damage may result from exposure to UV or sunlight. In one embodiment, the method comprises administering to the subject a composition comprising a therapeutically effective amount of the compounds described herein. In another embodiment, the composition is administered topically to an eye. In another embodiment, the subject is at high risk for an ophthalmic condition. The term “high risk” as used herein means a subject who shows at least one sign of possibly developing an ophthalmic condition, such as the ophthalmic conditions described herein. Signs indicating possible development of an ophthalmic condition include, for example, if the subject is suffering from diabetes, hypertension, arteriosclerosis, exhibits macular drusen, or smokes tobacco, or any other precursor to an ophthalmic condition. In yet another embodiment, the administration precedes exposure to sunlight and/or UV light.

### Further Information

The term “treating” is used to refer to either prophylactic and/or therapeutic treatments. In therapeutic applications, the compositions are administered to a patient already suffering from a disease, condition or disorder, in an amount sufficient to cure, at least partially arrest the symptoms of the disease, disorder or condition, or otherwise relieve to some extent one or more of the symptoms of the disease, condition or disorder being treated. Amounts effective for this use will depend on the severity and course of the disease, disorder or condition, previous therapy, the patient's health status and response to the drugs, and the judgment of the treating physician. In prophylactic applications, compositions containing the compounds described herein are administered to a patient susceptible to or otherwise at risk of a particular disease, disorder or condition.

In this use, the precise amounts also depend on the patient's state of health, weight, and the like. The term “therapeutically effective amount” includes both therapeutically effective amounts and prophylactically effective amounts. One non-limiting example of a prophylactic application is the administration of eye drop formulations of a compound of Formula I, II, IIIa, IIIb, IV or V prior to exposure to intense sunlight, e.g., prior to going to the beach or for extended outdoor activity.

In the case wherein the patient's condition does not improve, upon the doctor's discretion the administration of the compounds is administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds is given continuously or temporarily suspended for a certain length of time (i.e., a “drug holiday”).

Once improvement of the patients conditions has occurred, a maintenance dose is administered if necessary. Subsequently, in some embodiments, the dosage or the frequency of administration, or both, are reduced, as a function of the symptoms, to a level at which the improved disease, disorder or condition is retained. In some embodiments, patients require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but is determined according to the particular circumstances surrounding the case, including, e.g., the specific agent being administered, the route of administration, the condition being treated, and the subject or host being treated. In general, however, doses employed for adult human treatment will typically be in the range of about 0.03 to about 30 mg per day. In other embodiments, the doses employed for adult human treatment is in the range of about 0.1 to about 15 mg per day. The desired dose is conveniently presented in some embodiments, in a single dose or as divided doses administered simultaneously (or over a short period of time) or at appropriate intervals, for example as two, three, four or more sub-doses per day.

### Combination Therapies

In certain instances, it is appropriate to administer the compounds of Formula I, IIIa, IIIb, IV or V described herein (or a pharmaceutically acceptable salt, ester, amide, prodrug, or solvate) in combination with another therapeutic agent. By way of example only, if one of the side effects experienced by a patient upon receiving one of the compounds herein is inflammation, then it is appropriate to administer an anti-inflammatory agent in combination with the initial therapeutic agent. Or, by way of example only, therapeutic effectiveness of the compounds of Formula I, II, IIIa, IIIb, IV or V, described herein are enhanced by administration of an adjuvant (i.e., by itself the adjuvant only has minimal therapeutic benefit, but in combination with another therapeutic agent, the overall therapeutic benefit to the patient is enhanced). Or, by way of example only, the benefit of experienced by a patient is increased by administering the compounds of Formula I, II, IIIa, IIIb, IV or V, described herein with another therapeutic agent (which also includes a therapeutic regimen) that also has therapeutic benefit. By way of example only, in a treatment for macular degeneration involving administration of the compounds of Formula I, II, IIIa, IIIb, IV or V, described herein, increased therapeutic benefit results by also providing the patient with other therapeutic agents or therapies for macular degeneration. In any case, regardless of the disease, disorder or condition being treated, the overall benefit experienced by the patient is simply additive of the two therapeutic agents or the patient experiences a synergistic benefit.

Specific, non-limiting examples of combination therapies include use of at least one of the compounds of Formula I, II, IIIa, IIIb, IV or V with nitric oxide (NO) inducers, statins, negatively charged phospholipids, anti-oxidants, minerals, anti-inflammatory agents, anti-angiogenic agents, matrix metalloproteinase inhibitors, carotenoids, 13-cis-retinoic acid, or a compound having the structure of Formula (A):

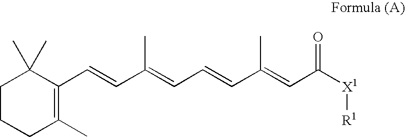

wherein
X¹ is selected from the group consisting of NR², O, S, CHR²;
R¹ is (CHR²)ₓ-L¹-R³, wherein
   x is 0, 1, 2, or 3; L¹ is a single bond or —C(O)—;
R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₂-C₇)cycloalkenyl, and a heterocycle.

In several instances, suitable combination agents fall within multiple categories (by way of example only, lutein is an anti-oxidant and a carotenoid). Further, in some embodiments, the compounds of Formula I, II, IIIa, IIIb, IV or V are administered with additional agents that provide benefit to the patient, including by way of example only, cyclosporin A.

In addition, the compounds of Formula I, II, IIIa, IIIb, IV or V are used in combination with procedures that provide additional or synergistic benefit to the patient, including, by way of example only, the use of extracorporeal rheopheresis (also known as membrane differential filtration), the use of implantable miniature telescopes, laser photocoagulation of drusen, and microstimulation therapy.

The use of anti-oxidants has been shown to benefit patients with macular degenerations and dystrophies. See, e.g., Arch. Opthalmol., 119: 1417-36 (2001); Sparrow, et al., J. Biol. Chem., 278:18207-13 (2003). Examples of suitable anti-oxidants that could be used in combination with the compounds of Formula I, II, IIIa, IIIb, IV or V include vitamin C, vitamin E, beta-carotene and other carotenoids, coenyme Q, lutein, butylated hydroxytoluene, resveratrol, a trolox analogue (PNU-83836-E), and bilberry extract.

The use of certain minerals has also been shown to benefit patients with macular degenerations and dystrophies. See, e.g., Arch. Ophihalmol., 119: 1417-36 (2001). Examples of suitable minerals that could be used in combination with at least one of the compounds of Formula I, II, IIIa, IIIb, IV or V include copper-containing minerals, such as cupric oxide (by way of example only); zinc-containing minerals, such as zinc oxide (by way of example only); and selenium-containing compounds.

The use of certain negatively-charged phospholipids has also been shown to benefit patients with macular degenerations and dystrophies. See, e.g., Shaban & Richter, Biol. Chem., 383:537-45 (2002); Shaban, et al., Exp. Eye Res., 75:99-108 (2002). Examples of suitable negatively charged phospholipids that could be used in combination with at least one of the compounds of Formula I, II, IIIa, IIIb, IV or V include cardiolipin and phosphatidylglycerol. In some embodiments, positively-charged and/or neutral phospholipids also provide benefit for patients with macular degenerations and dystrophies when used in combination with the compounds of Formula I, II, IIIa, IIIb, IV or V.

The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-Bcarotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a very high number of apoptotic photoreceptor cells, while the group with high zeaxanthin concentrations had minimal damage. Examples of suitable carotenoids for in combination with at least one of the compounds of Formula I, II, IIIa, IIIb, IV or V include lutein and zeaxanthin, as well as any of the aforementioned carotenoids.

Suitable nitric oxide inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the endothelium, and has been identified as nitric oxide or a closely related derivative thereof (Palmer et al, Nature, 327:524-526 (1987); Ignarro et al, Proc. Natl. Acad. Sci. USA, 84:9265-9269 (1987)).

Statins serve as lipid-lowering agents and/or suitable nitric oxide inducers. In addition, a relationship has been demonstrated between statin use and delayed onset or development of macular degeneration. G. McGwin, et al., British Journal of Opthalmology, 87:1121-25 (2003). Statins can thus provide benefit to a patient suffering from an ophthalmic condition (such as the macular degenerations and dystrophies, and the retinal dystrophies) when administered in combination with the compounds of Formula I, II, IIIa, IIIb, IV or V. Suitable statins include, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

Suitable anti-inflammatory agents with which the compounds of Formula I, II, IIa, IIIb, IV or V are used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, or Celebrex™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

In some embodiments, suitable matrix metalloproteinases (MMPs) inhibitors are administered in combination with the compounds of Formula I, II, IIIa, IIIb, IV or V in order to treat ophthalmic conditions or symptoms associated with macular or retinal degenerations. MMPs hydrolyze most components of the extracellular matrix. These proteinases play a central role in many biological processes such as normal tissue remodeling, embryogenesis, wound healing and angiogenesis. However, excessive expression of MMP has been observed in many disease states, including macular degeneration. Many MMPs have been identified, most of which are multidomain zinc endopeptidases. Representative examples of MMP Inhibitors include Tissue Inhibitors of Metalloproteinases (TIMPs) (e.g., TIMP-1, TIMP-2, TIMP-3, or TIMP-4), α₂-macroglobulin, tetracyclines (e.g., tetracycline, minocycline, and doxycycline), hydroxamates (e.g., BATIMASTAT, MARIMISTAT and TROCADE), chelators (e.g., EDTA, cysteine, acetylcysteine, D-penicillamine, and gold salts), synthetic MMP fragments, succinyl mercaptopurines, phosphonamidates, and hydroxaminic acids. Examples of MMP inhibitors that are used in combination with the compounds of Formula I, II, IIIa, IIIb, IV or V include, by way of example only, any of the aforementioned inhibitors.

The use of antiangiogenic or anti-VEGF drugs has also been shown to provide benefit for patients with macular degenerations and dystrophies. Examples of suitable antiangiogenic or anti-VEGF drugs that could be used in combination with at least one of the compounds of Formula I, II, IIIa, IIIb, IV or V include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone-ru486), subtenon triamcinolone acetonide, intravitreal crystalline triamcinolone acetonide, Prinomastat (AG3340-synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis).

Other pharmaceutical therapies that have been used to relieve visual impairment can be used in combination with at least one of the compounds of Formula I, II, IIIa, IIIb, IV or V. Such treatments include but are not limited to agents such as Visudyne™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEtema Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), C and 5 (Acuity Pharmaceuticals), ranibizumab (Genentech), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group pic), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporin A. See U.S. Patent Application Publication No. 20040092435.

In any case, the multiple therapeutic agents (one of which is the compounds of Formula I, II, IIIa, IIIb, IV or V described herein) are optionally administered in any order or even simultaneously. If simultaneously, the multiple therapeutic agents are optionally provided in a single, unified form, or in multiple forms (by way of example only, either as a single pill or as two separate pills). Optionally, one of therapeutic agents is given in multiple doses, or both are given as multiple doses. If not simultaneous, the timing between the multiple doses varies, in some embodiments, from more than zero weeks to less than four weeks. In addition, the combination methods, compositions and formulations are not to be limited to the use of only two agents; we envision the use of multiple therapeutic combinations. By way of example only, the compounds of Formula I, II, IIIa, IIIb, IV or V are provided with at least one antioxidant and at least one negatively charged phospholipid; or the compounds of Formula I, II, IIIa, IIIb, IV or V are provided with at least one antioxidant and at least one inducer of nitric oxide production; or the compounds of Formula I, II, IIIa, IIIb, IV or V are provided with at least one inducer of nitric oxide productions and at least one negatively charged phospholipid; and so forth.

In addition, for example, the compounds of Formula I, II, IIIa, IIIb, IV or V are used in combination with procedures that provide additional or synergistic benefit to the patient. Procedures to relieve visual impairment include but are not limited to ‘limited retinal translocation’, photodynamic therapy (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

Further combinations that are optionally used to benefit an individual include using genetic testing to determine whether that individual is a carrier of a mutant gene that is known to be correlated with certain ophthalmic conditions. By way of example only, defects in the human ABCA4 gene are thought to be associated with five distinct retinal phenotypes including Stargardt disease, cone-rod dystrophy, age-related macular degeneration and retinitis pigmentosa. In addition, an autosomal dominant form of Stargardt Disease is caused by mutations in the ELOV4 gene. See Karan, et al., Proc. Nail. Acad. Sci. (2005). Patients possessing any of these mutations are expected to find therapeutic and/or prophylactic benefit in the methods described herein.

### Kits/Articles of Manufacture

For use in the applications described herein, kits and articles of manufacture are also described herein. The terms “kit” and “article of manufacture” are used as synonyms. The kit can include a carrier, package, or container that is compartmentalized to receive one or more containers such as vials, tubes, and the like, each of the container(s) including one of the separate elements to be used in a method described herein. Preferably, containers (e.g., vials) containing an effective amount of a compound as described herein are light-proof have a tight seal. For example, the container(s) can include one of the eye drop formulations described herein, i.e., an eye drop formulation comprising an effective amount of a compound of Formula I, II, IIIa, IIIb, IV or V as described herein. In one embodiment the kit includes a dispenser containing a photo-protective ointment, for example, sunscreen, sun block, photo-protective clothing, photo-protective wash, or the like, or any other topical composition that protects against UVA and/or UVB radiation by providing some level of sun protection factor.

Suitable containers include, for example, bottles, vials, syringes, and test tubes. The containers can be formed from a variety of materials such as glass or plastic. Preferably, the container protects against certain wavelengths of light and prolonged high temperature, and/or the ingress of air. Preferably the container is a sealed, light-proof container.

The articles of manufacture provided herein contain packaging materials. Packaging materials for use in packaging pharmaceutical products include, by way of example only U.S. Pat. Nos. 5,323,907, 5,052,558 and 5,033,252. Examples of pharmaceutical packaging materials include, but are not limited to, blister packs, bottles, tubes, pumps, bags, vials, light-tight sealed containers, syringes, bottles, and any packaging material suitable for a selected formulation and intended mode of administration and treatment. A wide array of topical formulations of the compounds and compositions provided herein are contemplated as are a variety of treatments for any of the diseases, disorders, or conditions associated with oxidative damage described herein.

Such kits optionally comprise a compound with an identifying description or label or instructions relating to its use in the methods described herein. For example, the kit optionally includes instructions for use comprising the steps of applying the eye drop formulation to the eye, before and/or during exposure to the sun or UV light, i.e., before and/or during prolonged exposure to the sun or UV light.

In some embodiments, a kit includes one or more additional containers, each with one or more of various materials desirable from a commercial and user standpoint for use of the eye drop formulations described herein. Non-limiting examples of such materials include, but are not limited to, container, vial and/or tube labels listing contents and/or instructions for use, and package inserts with instructions for use. A set of instructions will also typically be included.

In certain embodiments, the eye drop formulations can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

In certain embodiments, the kit can additionally contain a UV radiation meter to indicate level of exposure to UV light. The UV radiation meter can be in the form of, for example, a UV sensitive strip, sensor, or a timer, or any other device to measure and/or indicate UV intensity. In some embodiments, the UV radiation meter can estimate maximum exposure time to sun and/or UV radiation and indicate risk for damage. In some embodiments, individual factors can be programmed into the UV radiation meter such as skin type, and sun protection factor of a photo-protective ointment used, if any. Such a kit is useful for sports and outdoor activities, such as hiking, skiing, snowboarding, volleyball, surfing, biking, fishing, or any other activity involving exposure to sun and/or UV light.

### EXAMPLES

The following ingredients, processes and procedures for practicing the methods disclosed herein correspond to that described above. The procedures below describe with particularity a presently preferred embodiment of the process for the detection and screening of modulators which reduce oxidative stress in the eye. The compounds and compositions described below are referred to as Topically Applied Preventatives of Peroxidation (TAPP).

### Example 1

### Synthesis of Compound 1 (TAPP1)

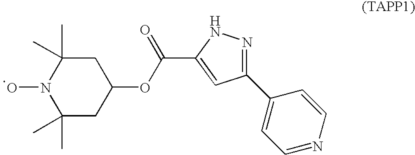

As presented in Scheme I (below), the synthesis begins with a Claisen condensation between 4-acetylpyridine and ethyl oxalate to afford the diketone. Condensation of the diketone with hydrazine hydrate provides the substituted pyrazole. Hydrolysis of the ester followed by coupling of the resulting acid to 4-hydroxy-1-oxyl-2,2,6,6-tetramethylpiperidine yields the desired product 1-oxyl-2,2,6,6-tetramethylpiperidin-4-yl 3-(pyridin-4-yl)-1H-pyrazole-5-carboxylate. Reagents and conditions: a. diethyl oxylate, NaOEt, THF, rt, 3 h; b. hydrazine hydrate, EtOH, 75° C., 12 h; c. 3 N HCl, 1,4-dioxane, 80° C., 12 h; d. CDI, DMF, 4-hydroxy-1-oxyl-2,2,6,6-tetramethylpiperidine, DBU, 12 h.

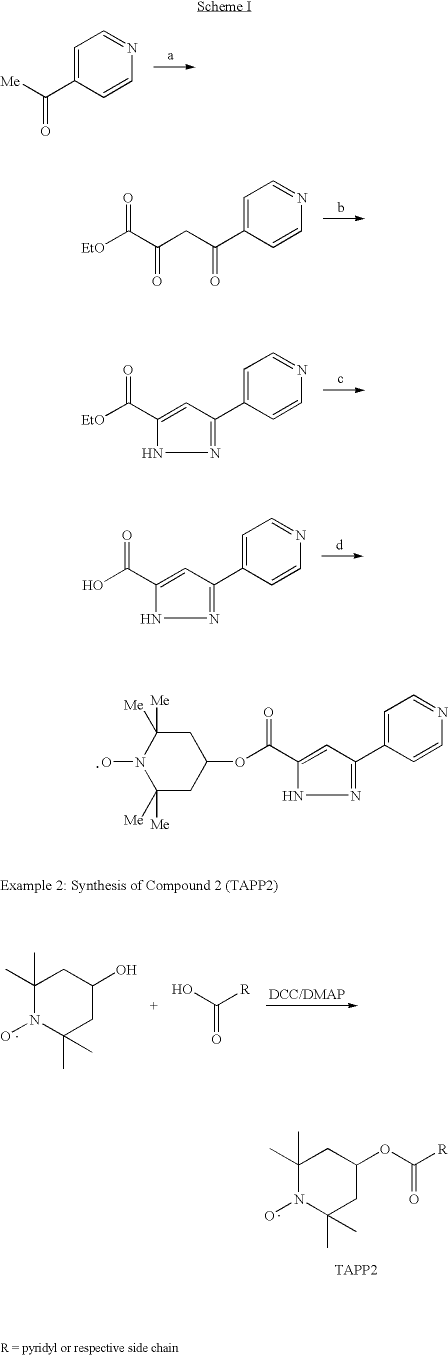

A mixture of 4-hydroxyl-2,2,6,6-tetramethylpiperidine (1 mmol), the appropriate carboxylic acid (1.1 mmol), 4-dimethylamino-pyridin (DMAP) (0.1 mmol), N,N′-dicyclohexylcarbodiimide (DCC) (1.1 mmol) in dichloromethane (CH₂CL) (10 ml) was stirred overnight at room temperature. The resulting suspension was filtered through celite, washed several times with CH₂CL. The filtrate was concentrated in vacuo and the residue was purified by flash chromatography and characterized by correct m/z in LCMS. Reagents and conditions: a. 4-hydroxyl-2,2,6,6-tetramethylpiperidine, appropriate carboxylic acid, DMAP, DCC, CH₂CL, rt, overnight

### Example 3

### Synthesis of Compound 3 (TAPP3)

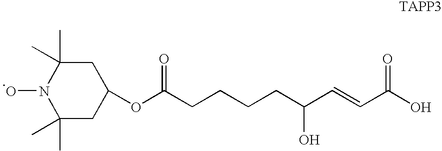

The generation of Compound 3 involves a 7-step synthesis followed by reduction, deprotection and oxidation.

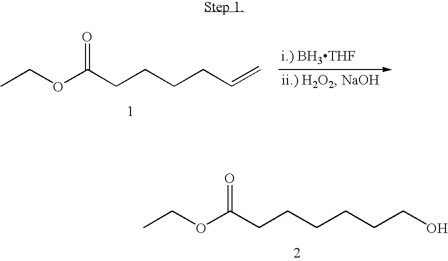

One molar borane solution (7.92 ml, 7.92 mmol) in tetrahydrofuran was added to ethyl 6-heptenoate (1) (3.713 g, 23.77 mmol) in 60 min at 0° C. After stirring for 2 hr at 0° C. and 2 hr at 25° C., the solution was hydrated with water. Next, 9.70 ml (24.46 mmol) of 30% hydrogen peroxide and 4.89 ml (4.89 mmol) of 1 M sodium hydroxide were added simultaneously at 10-22° C. The oxidized solution was diluted with 50 ml of ether, and the layers were separated. The aqueous layer was extracted with three 20 ml portions of ether, then saturated with potassium carbonate, and extracted with 250 ml of tetrahydrofuran. The organic layer and ether extracts were dried over magnesium sulfate and concentrated. The resulting oil was further purified by flash chromatography (eluent: hexane/ethyl acetate 2:1) to give 2.726 g (15.65 mmol, 67%) of (2) as a colourless oil. ¹H NMR (250 MHz, CDCl₃): δ=1.17 (3H, t, J=7.1 Hz), 1.26 (4H, br. m), 1.37 (2H, br. m), 1.51 (2H, br. m), 2.09 (1H, s), 2.25 (1H, d, J=7.3 Hz), 2.28 (1H, d, J=7.3 Hz), 3.35 (1H, d, J=6.5 Hz), 3.38 (1H, d, J=6.5 Hz), 4.04 (2H, q, J=7.1 Hz) ppm. See e.g, Herbert C. Brown and Kestutis A. Keblys; J. Am. Chem. Soc. 1964, 86, 1795-1801.

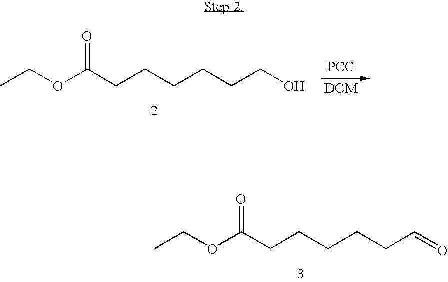

To a mixture of pyridinium chlorochromate (6.745 g, 31.29 mmol) and molecular sieves (3A, 2.5 g) in dry dichloromethane (100 ml), 2.726 g (15.65 mmol) ethyl 7-hydroxyheptenoate (2) was added at 0° C. The reaction mixture was stirred at room temperature until the reaction was complete (2 hours, TLC control). The solvent was evaporated and the residue was filtered through a small pad of silica gel using hexane/ethyl acetate 5:1 solvent mixture as eluent. The filtrate was concentrated to give the virtually pure aldehydes (3). Yield: 2.067 g (12.00 mmol, 77%) colourless oil. ¹H NMR (250 MHz, CDCl₃): δ=1.25 (3H, t, J=7.1 Hz), 1.37 (2H, br. m), 1.65 (4H, br. m), 2.28 (1H, d, J=7.6 Hz), 3.32 (1H, d, J=7.6 Hz), 4.13 (2H, q, J=7.1 Hz), 9.77 (1H, s) ppm; ¹³C NMR (63 MHz, CDCl₃): δ=14.37, 21.84, 24.54, 24.78, 28.72, 34.19, 43.78, 60.38, 173.64, 202.50 ppm.

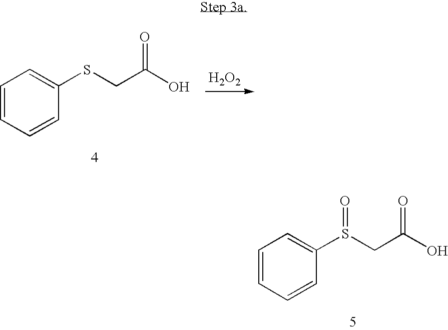

Phenylmercaptoacetic acid (4) (2.000 g, 11.89 mmol) in distilled water (10 ml) was slightly warmed to give a biphasic mixture. The rapidly stirred mixture was heated to 65° C. and then 30% hydrogen peroxide (1.35 ml, 11.89 mmol) was added slowly in several portions. Heating was needed to maintain the temperature at 65-70° C. since the reaction was only moderately exothermic. Starch-iodide paper was used to test when each portion of hydrogen peroxide had completely reacted before adding the next portion. A clear and homogeneous solution resulted after 3 hr and it was further heated until negative starch-iodide test showed no more hydrogen peroxide being present. When the reaction was over the mixture was allowed to cool to room temperature and water was removed under vacuum, on a water bath heated to 60° C. Digestion of the obtained clear syrup with hot toluene (15 ml) for a few minutes gave essentially pure phenylsulphinylacetic acid (5), which was filtered and washed with toluene (15 ml) and dried. Yield: 1.963 g (90%), Mp: 110-112° C. (Lit.: 108-111° C.). ¹H NMR (250 MHz, DMSO-d₆): δ=3.78 (1H, d, J=14.3 Hz), 4.01 (1H, d, J=14.3 Hz), 7.58 (3H, m), 7.72 (2H, m), 13.17 (1H, br. s) ppm; ¹³C NMR (63 MHz, DMSO-d₆): δ=61.28, 124.26, 129.29, 131.24, 143.74, 166.80 ppm. See e.g., Herman S. Schultz, Harlan B. Freyermuth, and Saul R. Buc J. Org. Chem. 1963, 28, 1140-1142. Derek Walker and Joseph Leib; Canadian Journal of Chemistry, 1962, 40, 1242-1248.

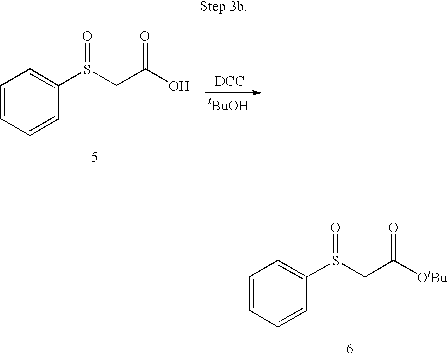

To the stirred mixture of phenylsulphinylacetic acid (5) (250 mg, 1.36 mmol) and N,N′-dicyclohexylcarbodiimide (336 mg, 1.63 mmol), dry tert-butanol (1.29 ml, 13.6 mmol) was added. When the reaction was complete (1 h, TLC control) the solvent was evaporated. To the reaction mixture was added H₂O (20 ml) and it was extracted with ethyl acetate (2×20 ml) and the combined organic layer was washed with saturated NaHCO₃, dried on MgSO₄ and concentrated in vacuum. The solid residue was purified by flash chromatography (eluent: hexane/ethyl acetate 5:1) to give 123 mg (0.52 mmol, 38%) of phenylsulphinylacetic acid tert-butyl ester (6) as white solid. ¹H NMR (250 MHz, CDCl₃): δ=1.40 (9H, s), 3.60 (1H, d, J=13.7 Hz), 3.80 (1H, d, J=13.6 Hz), 7.54 (3H, m), 7.71 (2H, m) ppm; ¹³C NMR (63 MHz, CDCl₃): δ=28.06, 62.79, 83.39, 124.58, 128.47, 131.81, 163.94 ppm.

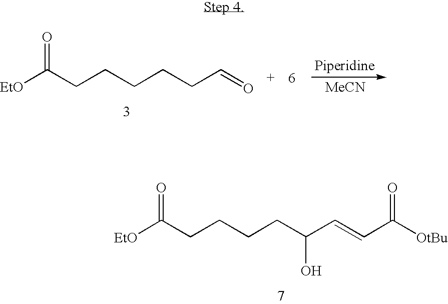

To the mixture of (6) (3.494 g, 14.54 mmol) and piperidin (1.423 ml) in acetonitrile (50 ml), (3) (2.256 g, 13.10 mmol) was added and stirred at room temperature. After the reaction was complete (14 h, TLC control) the reaction mixture was concentrated in vacuo and the oil rest was further purified by column chromatography (eluent: ethyl-acetate/hexane 1:5). Yield: 2.684 g (9.37 mmol, 72%). ¹H NMR (250 MHz, CDCl₃): δ=1.24 (3H, t, J=7.2 Hz), 1.47 (9H, s), 1.57 (6H, br. m), 2.28 (1H, d, J=7.3 Hz), 2.31 (1H, d, J=7.3 Hz), 4.11 (2H, q, J=7.1 Hz), 4.26 (1H, br.s), 5.92 (1H, d, 3J=15.6 Hz), 6.80 (1H, dd, J=15.6 Hz, J=15.6 Hz) ppm; ¹³C NMR (63 MHz, CDCl₃): δ=14.40, 24.84, 24.91, 28.28, 34.31, 36.37, 60.48, 70.98, 80.66, 122.26, 148.99, 166.03, 173.85 ppm.

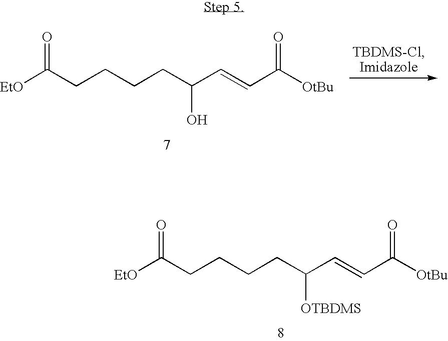

To the stirred solution of (7) (2.684 g, 9.37 mmol) and tert-butyldimethylsilyl chloride (1.695 g, 11.25 mmol) in DCM (120 ml), imidazole (1.595 g, 23.43 mmol) was added in one portion and stirred under Ar atmosphere. After the reaction was complete (5 h at RT, TLC control), DCM (50 ml) and water (50 ml) were added. The layers were separated and the organic layer was extracted three more times with brine, dried over MgSO₄, filtered and concentrated in vacuum. The oil rest was further purified by column chromatography (eluent: ethyl-acetate/hexane 1:5). Yield: 2.864 g (76%). ¹H NMR (250 MHz, CDCl₃): δ=0.34 (3H, s), 0.48 (3H, s), 0.89 (9H, s), 1.25 (3H, t, J=7.1 Hz), 1.49 (9H, s), 1.57 (6H, br. m), 2.29 (2H, t, J=7.3 Hz), 4.12 (2H, q, J=7.1 Hz), 4.27 (1H, q, J=5.5 Hz), 5.86 (1H, d, J=15.6 Hz), 6.77 (1H, dd, J=15.5 Hz, J=4.9 Hz) ppm.

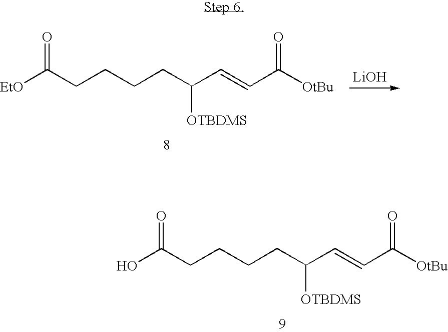

To the solution of (8) (192 mg, 0.48 mmol) in THF (2 ml) LiOH.H₂O (20 mg, 0.48 mmol) was added and stirred at RT. When the reaction was complete the solvent was evaporated in vacuum. To the residue, ethyl-acetate (5 ml) and water (5 ml) was added and the solution was acidified to pH 5 by slowly adding concentrated citric acid solution. Then the organic layer was separated and the water phase was extracted with ethyl-acetate (2×5 ml). The combined organic fraction was dried over MgSO₄, filtered and the solvent was evaporated in vacuo. The product was used without further purification. Yield: 109 mg (61%). ¹H NMR (250 MHz, CDCl₃): δ=0.01 (3H, s), 0.03 (3H, s), 0.88 (9H, s), 1.47 (9H, s), 1.54 (6H, br. m), 2.27 (2H, t, J=7 Hz), 4.25 (1H, q, J=5 Hz), 5.84 (1H, br. d, J=15 Hz), 6.76 (1H, br. dd, J=15.7 Hz, J=5.1 Hz), 9.37 (1H, br. s) ppm.

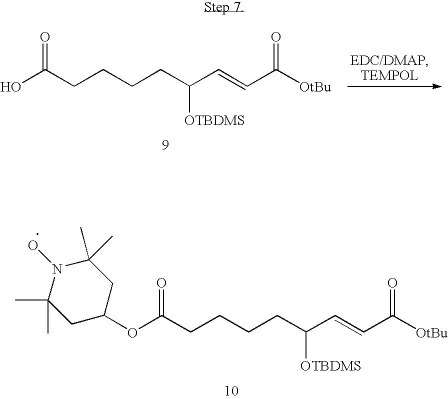

To the stirred mixture of (9) (1.252 g, 3.36 mmol), 4-hydroxyl-2,2,6,6-tetramethylpiperidine (695 mg, 4.03 mmol) and 4-dimethylaminopyridine (493 mg, 4.03 mmol) in dichloromethane (10 ml), 1-(3-dimethylaminopropyl)-3-ethylcarbodiimide (707 ul, 4.03 mmol) in dichloromethane (2 ml) was added drop wise. The reaction was stirred at RT for 48 h, concentrated in vacuum and the residue was purified by flash chromatography (eluent: hexan/diethyl ether 3:1) to afford the ester 10 (1.049 g, 59%).

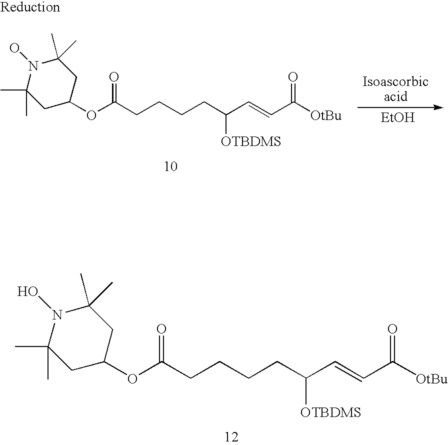

To the solution of (10) (105 mg, 0.27 mmol) in ethanol (1 ml), isoascorbic acid in water (200 μl) was added and stirred at room temperature until the reaction completed (TLC control, ca. 10 min). The solvent was removed under reduced pressure. The residue was dissolved in ethyl acetate (2 ml) and water (2 ml). The layers were separated and the water phase was extracted 3 more times with 2 ml ethyl acetate. The combined organic phase was dried on MgSO₄, filtered and concentrated in vacuum to afford 103 mg (98%) yellow oil. ¹H NMR (250 MHz, CDCl₃): δ=0.32 (3H, s), 0.34 (3H, s), 1.20 (9H, s), 1.48 (12H, s), 1.78 (9H, s), 1.62-1.95 (br. m), 2.14 (4H, m), 2.20 (1H, m), 2.56 (2H, t, J=7.6 Hz), 4.04 (3H, m), 4.56 (1H, q, J=5.4 Hz), 5.33 (1H, br. m), 6.14 (1H, d, J=15.6 Hz), 7.07 (1H, dd, J=15.6 Hz, J=4.9 Hz) ppm; ¹³C NMR (63 MHz, CDCl₃): δ=−6.07, −5.72, 16.95, 19.25, 23.13, 23.75, 24.43, 26.96, 28.48, 33.29, 35.79, 57.94, 65.29, 70.21, 79.05, 120.47, 124.26, 148.27, 164.80, 171.91 ppm.

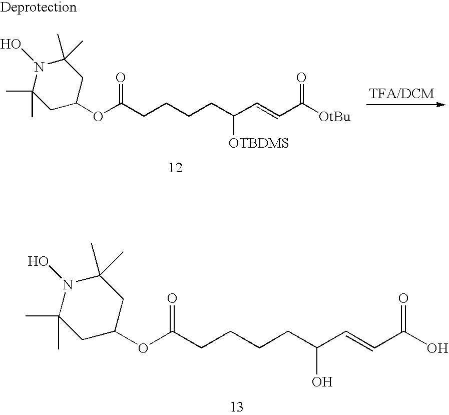

Deprotection of (12) with TFA in DCM (1:1) gave a mixture of products which was purified by chromatography (eluent hexan/THF 1:1 on column chromatography and preparative TLC).

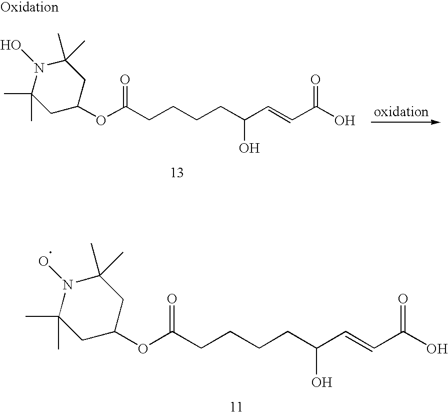

One-electron oxidations of the separated and purified (13) were made to obtain the radical target compound using 12 and Ag₂O. Oxidation with Ag₂O in dry ether gave only decomposition products after 3 days while giving a silver mirror on the glass wall of the flask. Oxidation of (13) with 12 in DCM afforded a multi component mixture containing a LC-MS peak having the molecular mass of the target compound.

The following compounds are prepared using the methods disclosed herein and appropriate starting materials and/or methods known in the art.

| Compd No. | Structure |
|---|---|
| 1 | 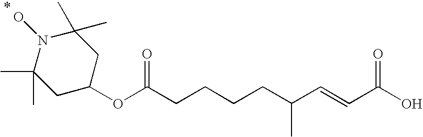 |
| 2 | 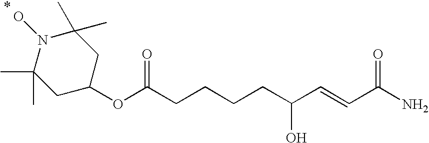 |
| 3 | 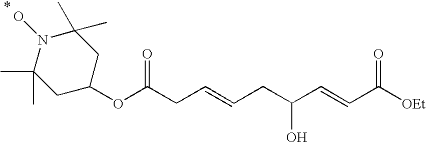 |
| 4 | 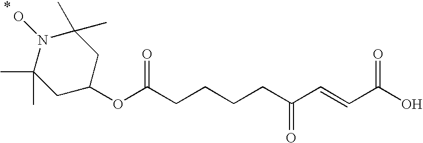 |
| 5 | 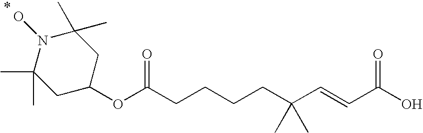 |
| 6 | 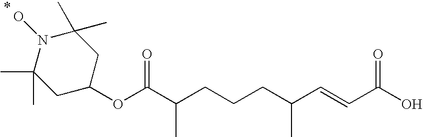 |
| 7 | 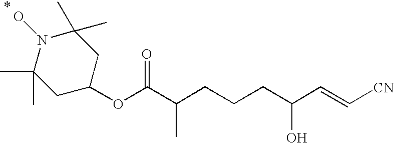 |
| 8 | 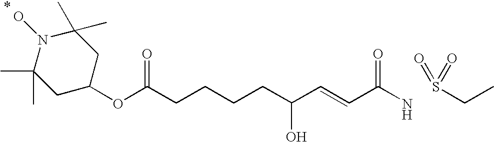 |
| 9 | 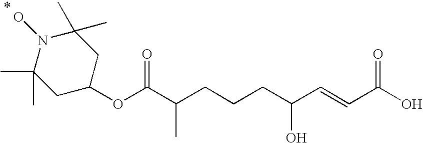 |
| 10 | 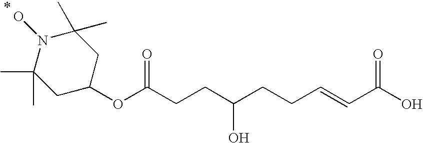 |
| 11 | 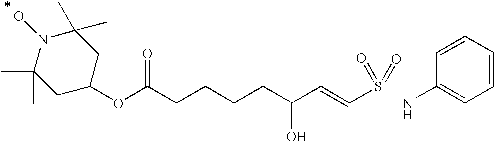 |
| 12 | 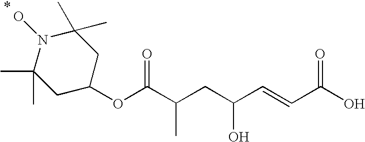 |
| 13 | 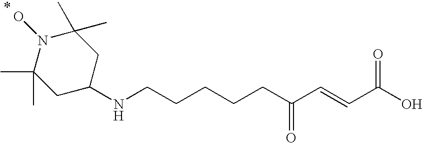 |
| 14 | 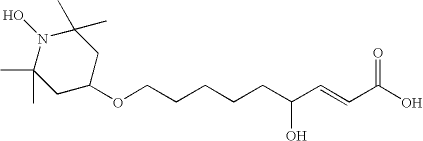 |
| 15 | 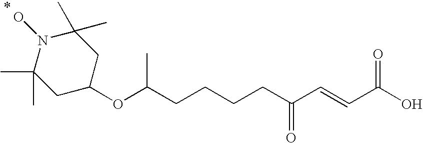 |
| 16 | 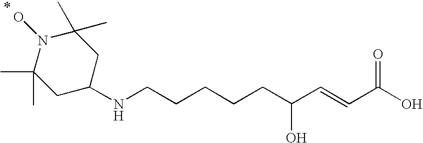 |
| 17 | 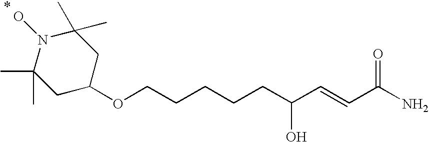 |
| 18 | 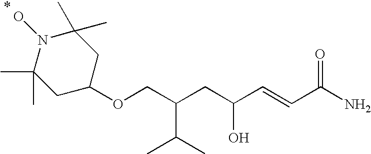 |
| 19 | 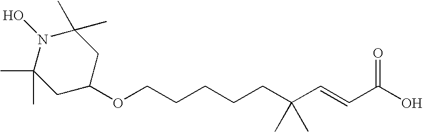 |
| 20 | 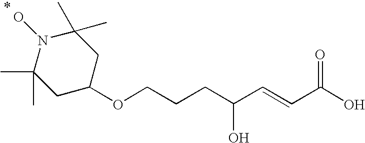 |
| 21 | 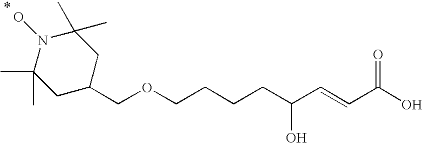 |
| 22 | 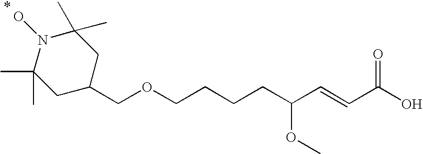 |
| 23 | 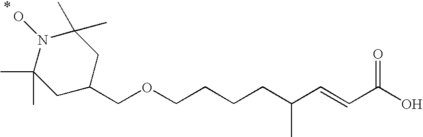 |
| 24 | 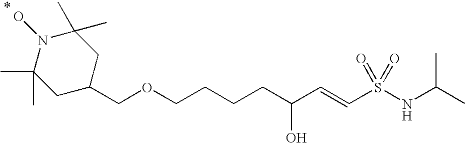 |
| 25 | 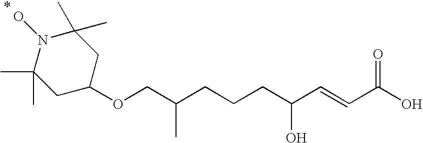 |
| 26 | 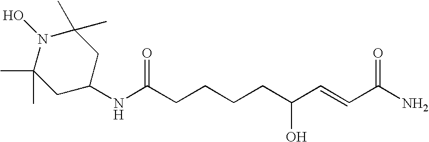 |
| 27 | 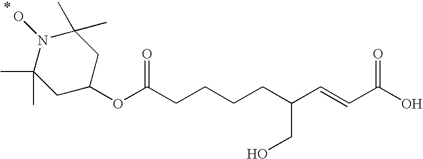 |
| 28 | 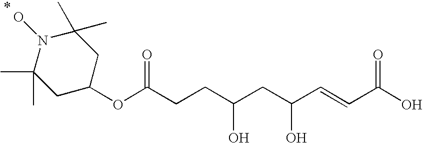 |
| 29 | 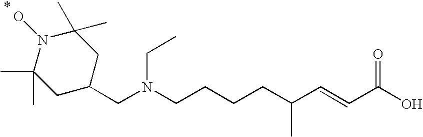 |
| 30 | 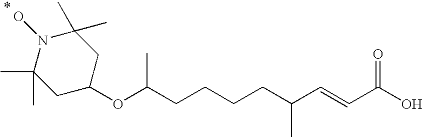 |
| 31 | 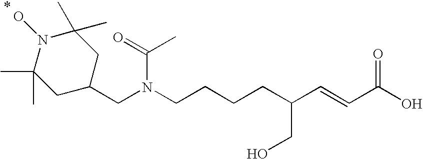 |
| 32 | 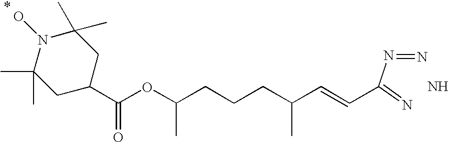 |
| 33 | 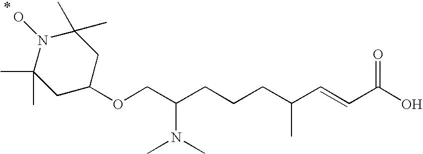 |
| 34 | 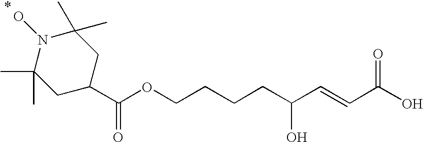 |
| 35 | 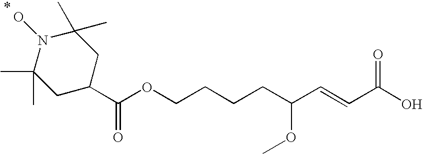 |
| 36 | 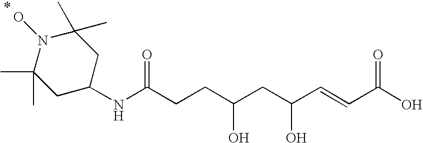 |
| 37 | 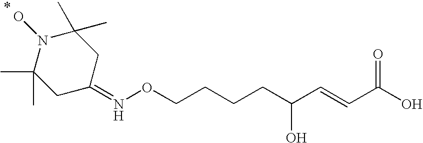 |
| 38 | 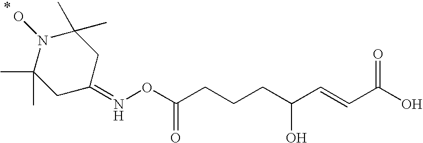 |
| 39 | 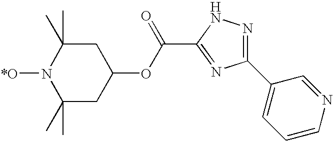 |
| 40 | 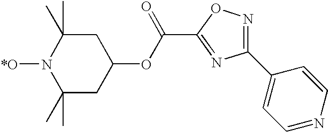 |
| 41 | 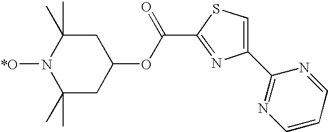 |
| 42 | 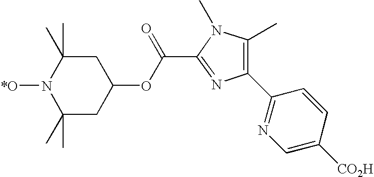 |
| 43 | 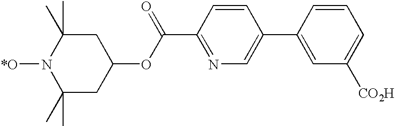 |
| 44 | 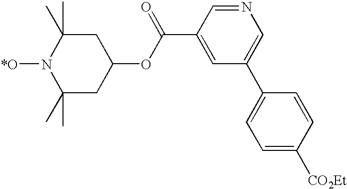 |
| 45 | 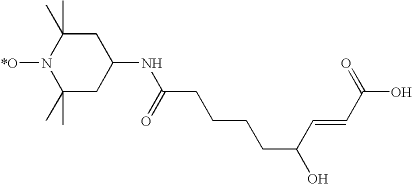 |
| 46 | 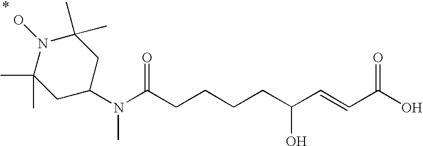 |
| 47 | 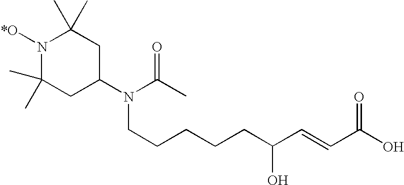 |
| 48 | 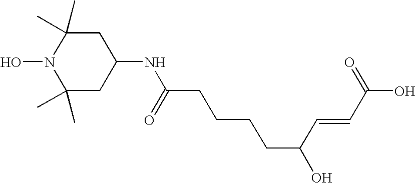 |

### Example 4

### Eyedrop Formulation of Compound I

To a suitable glass vessel is added compound of Formula I (1.0 mg/mL), sterile dextran (0.1%, w/v), hydroxymethylcellulose (0.3%, w/v), propylene glycol (0.3%, w/v), polyethylene glycol 400 (0.4%, w/v), and saline solution (0.001% potassium chloride, sodium borate and sodium chloride). The resulting mixture is mixed until a clear solution is formed. The clarified solution is used in the indicated treatment studies.

### Example 5

### Preparation of an Ophthalmic Formulation of Compound I Containing a Cyclodextrin

To a suitable glass vessel is added compound of Formula I (10 mg/mL), and hydroxypropyl-p-cyclodextrin (45%, w/v) or hydroxypropyl-γ-cyclodextrin (45%, w/v). The resulting mixture is stirred until the compound is optimally solubilized. This preparation is used in the indicated treatment studies

### Example 6

### Preparation of an Ophthalmic Formulation of Compound I for Human Trials

To a suitable glass vessel is added compound of Formula I (10 mg/mL), hydroxypropyl-β-cyclodextrin (45% w/v), and glycerin (0.5% w/v). The resulting mixture is stirred until the compound is optimally solubilized. Edetate disodium (0.05% w/v), benzalkonium chloride (0.01% w/v) are added and the mixture is diluted with saline (0.001% potassium chloride, sodium borate and sodium chloride). Hydrochloric acid and/or sodium hydroxide are added (to adjust pH). This preparation is used in the indicated human trials.

### Example 7

### In Vitro Assays of Anti-Oxidant Activity

Synthesized compounds are evaluated for antioxidant activity using a commercially available assay kit. This assay relies on the ability of the test compound to inhibit the oxidation of ABTS (2,2′,azino-di-[3-ethylbenzthiazoline-6-sulfonate]) to ABTS^{•+} radical cation by metmyoglobin. Experiments are performed according to procedure provided by the manufacture (Cayman Chemical Company, Ann Arbor, Mich.). Briefly, 50 μM of compound of Formula I, II or IIIa or IIIb (in methanol) are mixed at room temperature with a solution containing ABTS and metmyoglobin. Hydrogen peroxide is added to activate metmyglobin to the ferrylmyoglobin radical, which in turn oxidizes ABTS to form ABTS^{•+}. The oxidized form of ABTS produces a green color which absorbs at 405 nm and 750 nm. Absorption at 750 nm is monitored over time for samples in the presence or absence of compositions from compounds of Formula I, II, IIIa or IIIb. Relative anti-oxidant activity is determined by comparing absorbance values in the absence of the test compound (control), taken as 0% anti-oxidant potency, to the absorbance in the presence of the test compound.

### Example 8

### Evaluation of Potential Lens Toxicity

The TAPP compositions are intended for topical delivery to the anterior portion of the eye. Therefore, analysis of potential toxicity to the lens is routinely performed. To determine effects on whole eyes, eyeglobes from mice aged 42 days are placed in perfusion culture overnight either with control solution (45% beta-cyclodextrin) or a compound from the TAPP compositions (in 45% β-cyclodextrin), at 37° C. overnight, and in 95% air/5% CO2. Morphological or anatomical effects are determined by comparison of TAPP-treated samples to samples incubated with either the TAPP vehicle or samples which are exposed (10 min treatment) to 100% ethanol (a positive control for lens toxicity). To determine effects of TAPP compositions on isolated lenses, intact lenses are dissected from wild-type mouse eyeglobes. The lens samples are incubated in DMEM media in a 96 well tissue culture plate. Groups of 3-6 lenses are incubated in 200 μl of media alone (negative control), 4% (2-hydroxypropyl)-β-cyclodextrin (carrier control), 8 mM hydrogen peroxide (H₂O₂, positive control for lens toxicity), 4 nM TAPP1-O (free radical form), 4 mM TAPP1-R (reduced form), and 4 mM of the two TAPP1-R hydrolyzed products (HP-1 and HP-2), respectively. β-cyclodextrin is included as a carrier control. The samples are incubated in 5% CO₂ incubator at 37 C for 2 days and toxicity is determined by microscopic examination.

### Example 9

### Treatment of Oxidative Stress: In Vitro and Ex Vivo Studies

The compound TAPP1 (C₁₈H₂₄N₄O₃), MW 344.18, is formulated as described above. A 40 mM solution of TAPP1 is used. To determine the susceptibility of TAPP1 to hydrolysis by corneal esterases, TAPP1 is incubated with anterior segments prepared from the eyeglobes of wild-type mice. The anterior segments are cultured in 0.5 ml of MEM media and TAPP1 is added to a final concentration of 1 mM. Samples are incubated at 37° C. for various periods. At 0 min, 1 min, 5 min, 15 min, 30 min, 60 min and 120 min, 20 μl aliquot samples are removed from the culture. The aliquots are mixed with an equal volume of ice-cold methanol, and incubated on ice for 10 min, followed by centrifugation at 25,000 g to precipitate proteins. TAPP content of the supernatant is analyzed by a capillary reverse phase C18 column. Specifically, 0.2 μl of the supernatant is injected onto Zorbax 300SB-C18 column, using a flow rate of 10 μl/min at 40° C., and the sample is eluted using a gradient of acetonitrile in water (5-100%). The relative quantity of the TAPP is determined by area integration of the TAPP elution peak. To determine effects on isolated retina, whole retina explants are used. Retinas are dissected from the posterior segments of enucleated mouse eyeglobes. Retinas are then placed in culture medium containing either TAPP1 or the TAPP1 vehicle. Intense light exposure (48 hours of ˜10,000 Lux) is used to produce ROS. The generated ROS will, in turn, stimulate the formation of oxidized arachadonic fatty acid molecular species, or isoprostanes. Isoprostanes are measured with a commercially available in vitro assay kit.

### Example 10

### Treatment of Oxidative Stress: In Vivo Studies in a Mouse Model for AMD

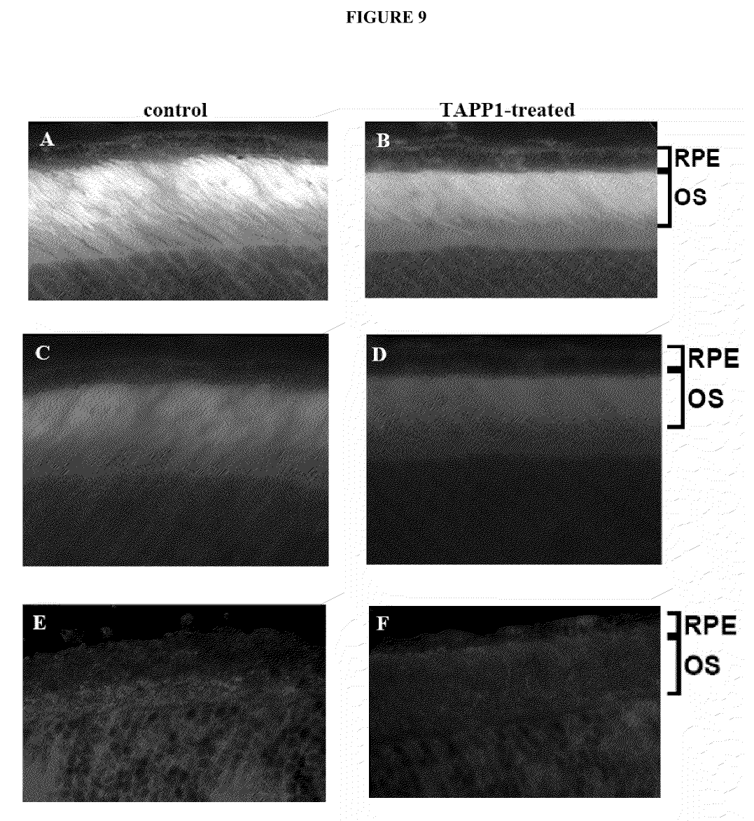

A mouse which lacks, or is deficient in, the superoxide dismutase 1 (SOD1) enzyme, is used as a model of oxidative stress. Previous investigations have shown that the SOD1-deficient mouse manifests a phenotype which is consistent with age-related macular degeneration (i.e., formation of drusen, thickening of Bruch's membrane and choroidal neovascularization; Imamura, et. al, Proc Natl Acad Sci, 103: 11282-11287, 2006). Following examination of appropriate biomarkers of oxidative stress, the SOD mutant mice are treated with an eye drop formulation containing 40 mM TAPP1 in β-cyclodextrin. The treatment protocol is one drop per day, 5 treatments per week for 6 weeks, for a total of 30 treatments. SOD mutant mice in the control group receive only the TAPP1 vehicle. Following the treatment regime, mice are sacrificed and eyeglobes are enucleated, fixed and prepared for histological examination. The presence of oxidative stress biomarkers (lipid hydroperoxides, LH; malondialdehyde, MDA; and nitrotyrosine, NT) in the tissue sections were evaluated by immunohistochemistry (FIG. 9).

### Example 11

### Measurement of LH, MDA and NT Immunoreactivity in SOD Mutant Mice

Tissue sections were fixed with 4% paraformaldehyde (or mixture of 4% paraformaldehyde+1% or 0.25% glutaraldehyde) and cryoprotected with 30% sucrose in 4% paraformaldehyde. The specimens are mounted in OCT and 10 um sections are cut. Sections are rehydrated with PBS and antigen retrieval is achieved by boiling in citrate buffer (0.01M, pH6.0, 3 times, 5 min each). Sections are permeabilised with ice-cold methanol, blocked with 5% goat serum in PBS (1 hr at RT) and then incubated overnight at 4° C. with the desired primary antibody (anti-LH, anti-MDA, or anti-NT) diluted in 1% goat serum (antibody dilutions range from 1:100-1:200). Sections are washed with PBS and then incubated with secondary antibody diluted in 1% normal donkey serum (1:2000 Alexa Fluor 546 donkey anti goat IgG at 2 mg/ml in the dark at RT). Samples are then washed with PBS and coverslipped with prolong antifade reagent.

### Example 12

### Measurement of Hvpopigmentation and Integrity of Retinal Vessels in SOD Mutant Mice

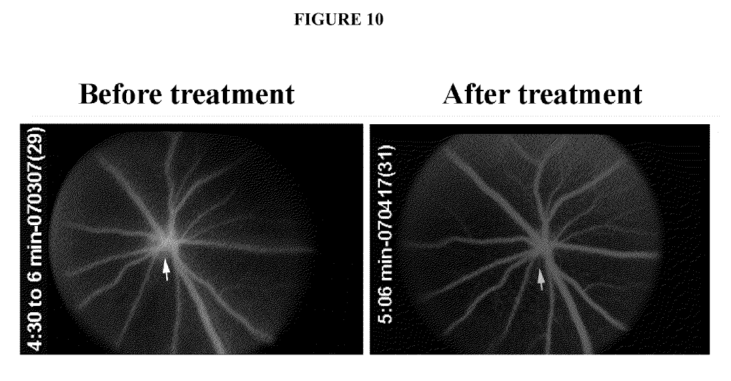

A Zeiss FF3 fundus camera with a camera back containing a barrier filter for fluorescein angiography (FA) is used. Animals are placed in a restraint built onto the fundus camera and injected with 25% sodium fluorescein (0.01 ml in sterile saline per 5-6 gm of body weight, i.p.). Immediately following the injection, a timer on the camera back is engaged so that elapsed time in seconds is recorded for each picture. The fluorescein images are recorded on Fujichrome Velvia RVP 100 color slide film. Analysis of SOD mutant mice (aged 186 days) reveals hypopigmentation and compromised retinal vessels around the optic disc. SOD mutant mice also demonstrate drusen-like deposits and hypopigmentation around the optic disc. Treatment with a TAPP1 composition (treatment regime described above) reduces the severity of hypopigmentation and improves the integrity of retinal vessels (i.e, there is reduced dye leakage around the optic disc in TAPP 1-treated SOD mutant mice) (FIG. 10).

### Example 13

### Determination of Dose Response and IC50 Values

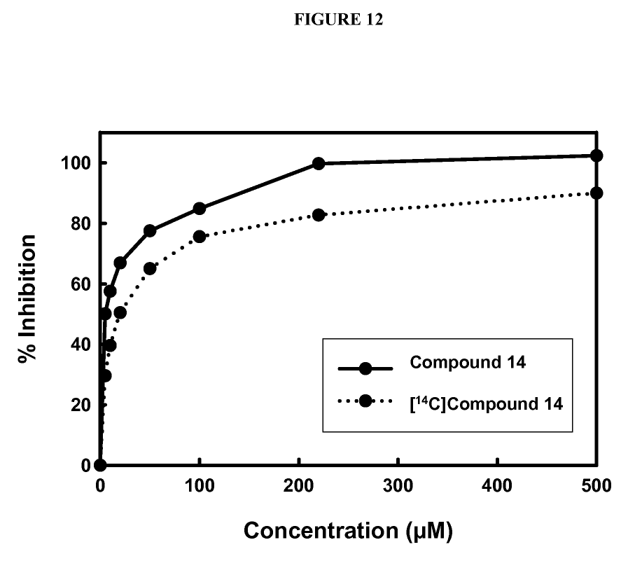

This anti-oxidation assay measures the potency of the test compound to inhibit the metmyoglobin-mediated oxidation of ABTS to ABTS^{•+} radical cation. [¹⁴C]Compound 14 (0, 5, 10, 20, 50, 100, 200, and 500 μM) was mixed with a solution containing 15 μM ABTS and 2.5 μM metmyoglobin at room temperature. Hydrogen peroxide at 80 μM was then added to the mixture to activate metmyoglobin to ferrylmyoglobin radical, which in turn oxidizes ABTS to ABTS^{•+}. The oxidized ABTS produced a green color. Anti-oxidant potency was calculated from the inhibition of ABTS^{•+} formation, based on the sample absorbance at 750 nm. [¹⁴C]Compound 14 demonstrated a dose-dependent anti-oxidation activity, with IC50 value of about 10 μM. As a reference, the anti-oxidation activity of non-radioactive Compound 14 is also shown (FIG. 12).

### Example 14

### Determination of Pharmacokinetics and Ocular Tissue Distribution in Mice

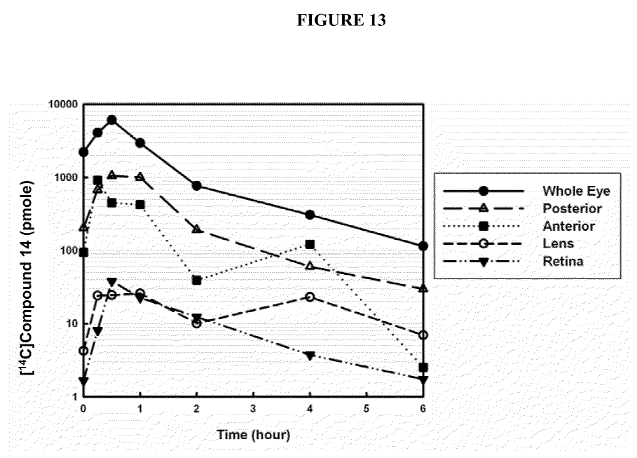

Seven ABCA4+/−/SOD+/− mice were used for this study. The mice were anesthetized and an ophthalmic formulation of Compound 14, prepared as described above, was administered topically to the eye. At each specified time post dosing (0 min, 15 min, 30 min, 1 hr, 2 hr, 4 hr and 6 hr), one mouse was sacrificed by cervical dislocation and both eyeballs were enucleated. Each eyeball was rinsed in 1 ml fresh PBS to wash off unabsorbed drug. The right eye was analyzed intact. From the left eye, the following tissues were harvested: lens, anterior segment, retina and posterior segment. Total radioactivity of all tissues was determined using a liquid scintillation analyzer. The data show that [¹⁴C]Compound 14 is taken into ocular tissues within 15 min post treatment. Peak drug concentration in all tissues occurred at ˜1 hr. The highest concentrations of [¹⁴C]Compound 14 were found in the anterior and posterior segments. Lower concentrations of [¹⁴C]Compound 14 were observed in the lens and retina. The pattern and time course for drug clearance from these tissues was also comparable (FIG. 13).

### Example 15

### Determination of Pharmacokinetics and Ocular Tissue Distribution in Rabbit

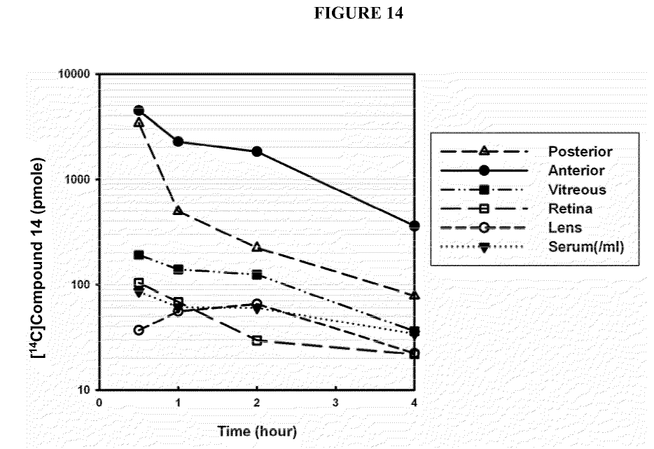

Four New Zealand White rabbits, age 3-4 months, weight 2.7 to 3.2 kg were used for this study. The rabbits were anesthetized and an ophthalmic formulation of Compound 14 was administered topically to the eye. At each specified time post dosing (0.5, 1, 2, and 4 hr), blood was collected from the ear vein of one rabbit and recovered serum volume was recorded. Rabbits were sacrificed by asphyxiation and both eyeballs were enucleated. Each eyeball was rinsed in 10 ml fresh PBS to wash off unabsorbed drug. The following tissues were harvested from each eye: anterior segment, lens, vitreous body, retina and posterior segment (without retina). Total radioactivity of each tissue and in serum was determined using a liquid scintillation analyzer (Packard Tri-Carb 2100TR). CPM was converted to DPM, and quantity of the drug (pmole) absorbed in each tissue was calculated based on specific activity (average DPM in tissues from left and right eyes). The serum level of the drug was determined as concentration (pmole/ml). [¹⁴C]Compound 14 was rapidly taken into ocular tissues (within ˜15 min) and peak drug concentration in all tissues occurred at ˜0.5 hr. The highest recovery of [¹⁴C]Compound 14 was observed in the anterior and posterior segments. Recovery of [¹⁴C]Compound 14 was approximately 10-fold lower in retina and lens, while the vitreous body contained an intermediate amount of [¹⁴C]Compound 14. The pattern and time course for drug clearance from these tissues was comparable (FIG. 14).

### Example 16

### In Vivo Studies in Mouse Model for Stargardt's Disease

The primary pathologic defect in Stargardt's disease is accumulation of toxic lipofuscin pigments in cells of the retinal pigment epithelium. This accumulation is responsible for the photoreceptor death and severe visual loss in Stargardt's patients.

Seven abcr(−/−) mice are administered eye drop compositions of placebo or Compound I. Eyes are enucleated 5 hours later. Tissues from these mice are analyzed biochemically for retinoids and lipofuscin pigments. Eyes from these mice are analyzed morphologically for lipofuscin in the retinal pigment epithelium and for degeneration of photoreceptors. Visual function in these mice is analyzed by electroretinography.

### Example 17

### Monitoring the Effectiveness of Ophthalmic Treatment, Therapies or Drugs

Assessing the effectiveness of treatments, therapies or drugs which have an effect on macular or retinal degenerations and dystrophies is a three step process which involves-1) taking initial measurements of a subject, such as the formation of drusen in the eye of the subject, size and number of geographic atrophy in the eye of the subject, measuring the levels of lipofuscin in the eye of the subject by measuring auto-fluorescence of A2E or lipofuscin and precursors of A2E, or measuring N-retinylidene-N-retinyl-phosphatidylethanolamine (A2PE) levels in the eye of the subject. 2) providing treatment, therapy or drug to the subject, 3) taking measurements of the formation of drusen in the eye of the subject, size and number of atrophic lesions in the eye of the subject, measuring the levels of lipofuscin in the eye of the subject by measuring the auto-fluorescence of A2E or lipofuscin and precursors of A2E, or measuring A2PE levels in the eye of the subject after step (2), and assessing results which indicate that the treatment, therapy or drug has a desired effect. A desired result includes reduced hypopigmentation of the fundus, increased integrity of retinal vessels (as measured by FA), a decrease or suppression of drusen formation and/or a reduction in lipofuscin levels in the eye of the subject as measured by auto-fluorescence of A2E or A2E precursors in the eye(s) of the subject. Reiteration of steps 2-3 is optionally administered with or without intervals of non-treatment. Subjects include but are not limited to mice and/or rats and/or human patients.

### Example 18

### Testing for the Efficacy of Compounds Which Reduce Oxidative Damage or Stress to Treat Macular Degeneration—TAPP1 As An Illustrative Compound

For pre-testing, all human patients undergo a routine opthalmologic examination including FA, measurement of visual acuity, electrophysiologic parameters and biochemical and rheologic parameters. Inclusion criteria are as follows: visual acuity between 20/160 and 20/32 in at least one eye and signs of AMD such as hypopigmentation of the fundus, the presence of large soft drusen, areolar atrophy, pigment clumping, pigment epithelium detachment, or subretinal neovascularization. Patients that are pregnant or actively breast-feeding children are excluded from the study.

Two hundred human patients diagnosed with macular degeneration, or who have progressive formations of A2E, lipofuscin, or drusen in their eyes are divided into a control group of about 100 patients and an experimental group of 100 patients. A composition comprising TAPP1 is administered to the experimental group on a daily basis. A placebo is administered to the control group in the same regime as a composition comprising TAPP1 is administered to the experimental group.

Administration of a composition comprising TAPP1 or placebo to a patient is topically to the eye at amounts effective to inhibit the development or reoccurrence of macular degeneration. Effective dosage amounts are in the range of from about 1-4000 mg/m² up to three times a day.

One method for measuring progression of macular degeneration in both control and experimental groups is the best corrected visual acuity as measured by Early Treatment Diabetic Retinopathy Study (ETDRS) charts (Lighthouse, Long Island, N.Y.) using line assessment and the forced choice method (Ferris et al. Am J Opthalmol, 94:97-98 (1982)). Visual acuity is recorded in logMAR. The change of one line on the ETDRS chart is equivalent to 0.1 logMAR. Further typical methods for measuring progression of macular degeneration in both control and experimental groups include use of visual field examinations, including but not limited to a Humphrey visual field examination, and measuring/monitoring the autofluorescence or absorption spectra of A2E and related toxic fluorophores (e.g., N-retinylidene-phosphatidylethanolamine and/or dihydro-N-retinylidene-N-retinyl-phosphatidylethanolamine (A2PE-H2)) in the eye of the patient. Autofluorescence is measured using a variety of equipment, including but not limited to a confocal scanning laser opthalmoscope. See Bindewald, et al., Am. J. Opthalmol., 137:556-8 (2004).

Additional methods for measuring progression of macular degeneration in both control and experimental groups include taking fundus photographs, observing changes in autofluorescence over time using a Heidelberg retina angiograph (or alternatively, techniques described in M. Hammer, et al. Opthalmologe 2004 Apr. 7 [Epub ahead of print]), and taking fluorescein angiograms at baseline, three, six, nine and twelve months at follow-up visits. Documentation of morphologic changes include changes in (a) drusen size, character, and distribution; (b) development and progression of choroidal neovascularization; (c) other interval fundus changes or abnormalities; (d) reading speed and/or reading acuity; (e) scotoma size; or (f) the size and number of the geographic atrophy lesions. In addition, Amsler Grid Test and color testing are optionally administered.

To assess statistically visual improvement during drug administration, examiners use the ETDRS (LogMAR) chart and a standardized refraction and visual acuity protocol. Evaluation of the mean ETDRS (LogMAR) best corrected visual acuity (BCVA) from baseline through the available post-treatment interval visits aids in determining statistical visual improvement.

To assess the ANOVA (analysis of variance between groups) between the control and experimental group, the mean changes in ETDRS (LogMAR) visual acuity from baseline through the available post-treatment interval visits are compared using two-group ANOVA with repeated measures analysis with unstructured covariance using SAS/STAT Software (SAS Institutes Inc, Cary, N.C.).

Toxicity evaluation after the commencement of the study includes check ups every three months during the subsequent year, every four months the year after and subsequently every six months. The toxicity evaluation includes patients using the composition comprising TAPP1 as well as the patients in the control group.

### Example 19

### Testing for the Efficacy of Compounds Which Reduce Oxidative Damage or Stress to Treat Open-Angle Glaucoma or Ocular Hypertension.—TAPP1 as an Illustrative Compound

For pre-testing, four parameters will be evaluated for all groups: Best corrected visual acuity, Optic disc cupping, visual fields and general perimetric indices and peripapillary retinal nerve fiber layer.

Every participant in the study, after giving his informed consent, will be evaluated by a senior ophthalmologist in a single office appointment. The appointment will include a visual acuity, complete ophthalmic examination, Humphrey perimetric visual field testing and peripapillary RNFL thickness measurement by OCT. After data collection, average +/−Standard deviation for the 4 parameters will be compared between the 3 groups. Student T-test and one-way ANOVA will be used for statistical analysis.

Inclusion Criteria: 18 years or older, clinical diagnosis of open-angle glaucoma (with or without pseudoexfoliation or pigment dispersion component) or ocular hypertension.

Exclusion Criteria: pregnancy, visual acuity less then 6/60

350 human patients diagnosed with glaucoma are divided into a control group of about 175 patients and an experimental group of 175 patients. An eye drop composition comprising TAPP1 is administered to the experimental group three times a day. A placebo is administered to the control group in the same regime as a composition comprising TAPP1 is administered to the experimental group.

Primary Outcome Measures: Reduction in Intraocular Pressure.

Secondary Outcome Measures: Visual Acuity; side effects

To assess statistically visual improvement during drug administration, examiners use the ETDRS (LogMAR) chart and a standardized refraction and visual acuity protocol. Evaluation of the mean ETDRS (LogMAR) best corrected visual acuity (BCVA) from baseline through the available post-treatment interval visits aids in determining statistical visual improvement.

To assess the ANOVA (analysis of variance between groups) between the control and experimental group, the mean changes in ETDRS (LogMAR) visual acuity from baseline through the available post-treatment interval visits are compared using two-group ANOVA with repeated measures analysis with unstructured covariance using SAS/STAT Software (SAS Institutes Inc, Cary, N.C.).

Toxicity evaluation after the commencement of the study includes check ups every three months during the subsequent year, every four months the year after and subsequently every six months. The toxicity evaluation includes patients using the composition comprising TAPP1 as well as the patients in the control group.

It is understood that the examples and embodiments described herein are for illustrative purposes only and that various modifications or changes in light thereof will be suggested to persons skilled in the art and are to be included within the spirit and purview of this application and scope of the appended claims.

1. A compound of Formula III or pharmaceutically acceptable salt, or pharmaceutically acceptable prodrug thereof: 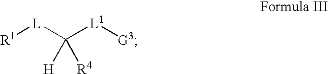
wherein,
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —OC(═O)—, —C(═O)O—, —OCH₂—, —CH₂O—, —NR⁶C(═O)—, —C(═O)NR⁶—, —NR⁶CH₂—, —CH₂NR⁶—, or an optionally substituted C₁-C₈alkylene;
L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl;
R⁶ is H, an optionally substituted C₁-C₄ alkyl group, —C(═O)R², and —SO₂N(R³)₂.

2. The compound of claim 1, wherein:
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —OC(═O)—, —C(═O)O—, —OCH₂—, —CH₂O—, or an optionally substituted C₁-C₈alkylene;
L¹ is a bond or an optionally substituted C₁-C₈alkylene;
G³ is selected from H, —CN, —CO₂H, —CO₂R², —C(═O)N(R³)₂, —C(═O)R², —N(R²)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl.

3. The compound of claim 1, wherein:
R¹ is N-oxyl-2,2,6,6-tetramethylpiperidin-4-yl, N-oxyl-2,2,6,6-tetramethylpiperidin-4-oximyl, or N-oxyl-2,2,5,5-tetramethylpyrrolidin-3-yl;
L is —NR⁶C(═O)—, —C(═O)NR⁶—, —NR⁶CH₂—, —CH₂NR⁶—, or an optionally substituted C₁-C₈alkylene;
L¹ is a bond or an optionally substituted C₁-C₈alkylene;
G³ is selected from H, —CN, —CO₂H, —CO₂R², —C(═O)N(R³)₂, —C(═O)R², —N(R³)₂, tetrazolyl, —NHS(═O)₂R², —S(═O)₂N(R³)₂, —OH, —OR², —C(═O)CF₃, —C(═O)NHS(═O)₂R², —S(═O)₂NHC(═O)R², —SR³, —NR³C(═NR³)NR³, optionally substituted aryl, and an optionally substituted heteroaryl;
each R² is independently an optionally substituted C₁-C₄ alkyl group or an optionally substituted aryl, or an optionally substituted heteroaryl;
each R³ is independently selected from H, an optionally substituted C₁-C₄ alkyl group, an optionally substituted aryl, and an optionally substituted heteroaryl;
R⁴ is H or —N(R⁵)₂;
each R⁵ is independently selected from H, or an optionally substituted C₁-C₄ alkyl;
each R⁶ is independently selected from H, an optionally substituted C₁-C₄ alkyl group, —C(═O)R², and —S(═O)₂N(R³)₂.

4. The compound of claim 1, wherein:
R¹ is N-oxyl-2,2,6,6-tetramethylpiperidin-4-yl.

5. The compound of claim 1, wherein:
R¹ is N-oxyl-2,2,6,6-tetramethylpiperidin-4-yl.

6. The compound of claim 5, wherein:
R⁴ is N(R⁵)₂; and
R⁵ is H.

7. The compound of claim 5, wherein:
R⁴ is H.

8. The compound of claim 4, wherein:
L¹ is an optionally substituted C₁-C₈alkylene optionally containing at least one unit of unsaturation.

9. The compound of claim 8, wherein:
L¹ is a bond, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH═CH—, or —CH₂CH₂CH₂CH₂CH(OH)CH═CH—.

10. The compound of claim 4, wherein:
-L¹-G³ is selected from H, —CH₃, —CH₂CH(CH₃)₂, —CH₂CO₂H, —CH₂CH₂CO₂H, —CH₂CH₂CH₂CH═CHCO₂H, —CH₂CH₂CH₂CH₂CH(OH)CH═CHCO₂H, —CH₂CONH₂, —CH₂CH₂CONH₂, —CH₂CH₂CH₂CH₂NH₂, —CH₂CH₂CH₂NC(═NH)NH₂, —CH₂OH, —CH₂CH₂SCH₃, —CH(OH)CH₃, —CH₂SH, —CH(CH₃)₂, —CH(CH₃)CH₂CH₃, —CH₂-imidazolyl, —CH₂-(1H-indol-3-yl), —CH₂-phenyl, —CH₂-(4-hydroxyphenyl).

11. The compound of claim 10, wherein:
G³ is selected from —CO₂H, —CO₂R² and tetrazolyl.

12. The compound of claim 2, wherein:
L is —OC(═O)—, —OCH₂—, or an optionally substituted C₁-C₈alkylene.

13. A compound selected from:
1-oxyl-2,2,6,6-tetramethylpiperidin-4-yl 2-(pyridin-2-yl)acetate;
1-oxyl-2,2,6,6-tetramethylpiperidin-4-yl 2-amino-3-phenylpropanoate;
4-(1-oxyl-2,2,6,6-tetramethylpiperidin-4-yloxy)-4-oxobutanoic acid; and
(E)-8-((N-oxyl-2,2,6,6-tetramethylpiperidin-4-yloxy)carbonyl)-4-hydroxyoct-2-enoic acid.

14. The compound of claim 3, wherein:
L is —NR⁶C(═O)—, —NR⁶CH₂— or an optionally substituted C₁-C₈alkylene.

15. A compound selected from:
(E)-9-((2,2,6,6-tetramethylpiperidin-1-oxyl)-4-aminyl)-9-oxo-4-hydroxynon-2-enoic acid;
(E)-9-((2,2,6,6-tetramethylpiperidin-1-oxyl)-4-amino-(N-acetyl))-4-hydroxynon-2-enoic acid.

16. A composition comprising a compound of claim 1 and an opthalmically acceptable excipient.

17. A method for reducing ophthalmic reactive oxygen species in a subject suffering from or at risk of suffering from an ophthalmic condition characterized by oxidative damage, comprising administering to the subject a composition comprising a therapeutically effective amount of a compound of claim 1.

18. The method of claim 17, wherein the ophthalmic condition is a vitreoretinal disease or condition.

19. The method of claim 17, wherein the ophthalmic condition is diabetic retinopathy, wet age-related macular degeneration, dry age-related macular degeneration, Stargardt' disease, macular edema, glaucoma, ocular hypertension, cataracts, corneal disorders or optic neuropathy.

20. The method of claim 17, wherein the administration is ophthalmic.
